(19) United States
(12) Patent Application Publication
(10) Pub. No.: US 2013/0289019 A1
(43) Pub. Date: Oct. 31, 2013

(54) METHODS OF TREATING BEHAVIORIAL AND/OR MENTAL DISORDERS

(71) Applicant: AMAZING GRACE, INC., Ashburn, VA (US)

(72) Inventor: David T. Chau, Ashburn, VA (US)

(73) Assignee: Amazing Grace, Inc., Ashburn, VA (US)

(21) Appl. No.: 13/870,739

(22) Filed: Apr. 25, 2013

Related U.S. Application Data
(60) Provisional application No. 61/639,000, filed on Apr. 26, 2012.

(51) Int. Cl. A61K 31/5513; A61K 31/4995; A61K 31/14; A61K 31/46; A61K 31/455

(52) U.S. Cl. CPC A61K 31/5513; A61K 31/46; A61K 31/455; A61K 31/14; A61K 31/4995
USPC 514/215; 514/220; 514/319; 514/252.16; 514/291; 514/356; 514/643; 514/424; 514/479; 514/278; 514/250; 514/343

(57) ABSTRACT

One embodiment of an aspect of the present invention is a method for lessening the symptoms of depression, anxiety, and post-traumatic stress disorder comprising the step of administering a therapeutically effective quantity of a cholinergic M1 receptor antagonist and a therapeutically effective quantity of one or more cholinomimetic agents to lessen the symptoms of depression, anxiety, and post-traumatic stress disorder. Typically, the cholinergic M1 receptor antagonist is selected from the group consisting of telenzepine, amytriptyline, biperiden, trihexyphenidyl, darifenacin, dicyclomine, and tiotropium. Another aspect of the present invention is directed to methods and compositions employing other therapeutic agents and combinations of therapeutic agents for emulating the theoretical pharmacological effects of the non-selective mAChR antagonist scopolamine. The invention also encompasses pharmaceutical compositions incorporating one or more therapeutic agents and a pharmaceutically acceptable carrier.

### CROSS-REFERENCES

This application claims the benefit of U.S. Provisional Patent Application Ser. No. 61/639,000 by David Chau, entitled “Methods of Treating Behavioral and/or Mental Disorders,” filed Apr. 26, 2012, the contents of which are hereby incorporated by reference in their entirety in this application.

### FIELD OF THE INVENTION

This invention is directed to methods and compositions for treating behavioral and/or mental disorders, including, but not limited to, depression, anxiety, post-traumatic stress disorder, substance abuse disorder, schizophrenia, eating disorders, obsessive-compulsive disorder, anxiety disorder, attention deficit hyperactivity, sleep disorders, decreased pleasure and motivation, and dysphoria (e.g., substance-induced or chemotherapy-induced dysphoria). In particular, this invention describes new treatments of depression, anxiety, and PTSD by administering a cholinergic M1 receptor antagonist, alone or in combination with a cholinomimetic that either elevates the level of acetylcholine or directly or indirectly activate cholinergic receptors other than the M1 receptor.

### BACKGROUND OF THE INVENTION

Behavioral and/or mental disorders, including, but not limited to, depression, anxiety, post-traumatic stress disorder (PTSD), substance abuse disorder, schizophrenia, eating disorders, obsessive-compulsive disorder, anxiety disorder, attention deficit hyperactivity, sleep disorders, decreased pleasure and motivation, and dysphoria (e.g., substance-induced or chemotherapy-induced dysphoria), affect a significant proportion of the population in most developed countries. These diseases and conditions are associated with a number of societal problems, including school failure, unemployment, disability, criminal activity, homelessness, and family breakup. In addition, the symptoms of the diseases and conditions are extremely troubling to the sufferers and disrupt lives, sometimes to the point that the sufferers attempt to or actually commit suicide. Although a number of drugs and pharmaceutical compositions are currently in use to treat these diseases and conditions, including monoamine oxidase inhibitors, selective serotonin reuptake inhibitors, tricyclic antidepressants, and antipsychotic agents, including phenothiazines, thioxanthenes, and other agents, these agents are frequently not well tolerated and compliance by patients with therapeutic regimes is frequently poor. A number of significant side effects are associated with many of these drugs, including, but not limited to, epigastric distress, constipation, dizziness, tachycardia, blurred vision, urinary retention, postural hypotension, weakness, fatigue, confusion, delirium, nausea, vomiting, sexual dysfunction, acute dystonia, akathisia, parkinsonism, neuroleptic malignant syndrome, and tardive dyskinesia.

Researchers at various academic institutions as well as the pharmaceutical industry have long been focused on the serotonin and norepinephrine systems (i.e., monoaminergic systems) as targets for antidepressant drugs. However, monoamine-based medications that do not have a cholinergic component have limited efficacy. First, ⅓ of all depressed patients do not respond to any current monoamine antidepressant. Second, patients' responses to monoamine medications vary widely, as clinicians frequently have to try several different such medications before they can identify a drug or drug combination that will work. Third, even if the “correct” monoamine-based medications are used, depression does not subside until after daily use for 3-4 weeks. Such trial and error approach and delayed therapeutic effects of currently available medications are recognized as major limitations, resulting in increased morbidity and risk of suicide for depressed patients.

Therefore, there is a need for improved methods and compositions to treat these diseases and conditions. Preferably, such improved methods and compositions are well tolerated and have fewer and less severe side effects, and thereby improve patient compliance with therapeutic regimens.

### SUMMARY OF THE INVENTION

One embodiment of an aspect of the present invention is a method for lessening the symptoms of depression, anxiety, and PTSD comprising the step of administering a therapeutically effective quantity of a cholinergic M1 receptor antagonist and a therapeutically effective quantity of one or more cholinomimetic agents to lessen the symptoms of depression, anxiety, and PTSD. Typically, the cholinergic M1 receptor antagonist is selected from the group consisting of telenzepine, amytriptyline, biperiden, trihexyphenidyl, darifenacin, dicyclomine, and tiotropium. This embodiment also includes a composition comprising a therapeutically effective quantity of a cholinergic M1 receptor antagonist, a therapeutically effective quantity of one or more cholinomimetic agents, and, optionally, a pharmaceutically acceptable carrier for use in lessening the symptoms of depression, anxiety, and PTSD. When present, the pharmaceutically acceptable carrier can be selected from the group consisting of a solvent, a buffer, a preservative, a solid filler, an excipient, a diluent, a dispersion medium, a coating, an antibacterial and/or antifungal agent, an isotonic agent, and an absorption-delaying agent.

In one alternative, the cholinomimetic comprises an acetylcholinesterase inhibitor. The acetylcholinesterase inhibitor is typically selected from the group consisting of:

(1) a phenanthrene derivative;

(2) tacrine;

(3) a carbamate derivative;

(4) a piperidine derivative;

(5) caffeine;

(6) huperzine;

(7) xanthostigmine;

(8) aminobenzoic acid;

(9) flavonoid;

(10) pyrrolo-oxazole;

(11) edrophonium;

(12) ladostigil;

(13) ungeremine;

(14) lactucopicrin; and

(15) coumarin.

When the acetylcholinesterase inhibitor is a phenanthrene derivative, typically the phenanthrene derivative is galantamine. When the acetylcholinesterase inhibitor is a carbamate derivative, typically the carbamate derivative is selected from the group consisting of rivastigmine, physostigmine, neostigmine, pyridostigmine, ambenonium, and demarcarium. When the acetylcholinesterase inhibitor is a piperidine, typically the piperidine is donezepil.

In another alternative, the cholinomimetic is a cholinergic muscarinic receptor agonist. Typically, the cholinergic muscarinic receptor is selected from the group consisting of piracetam, bethanechol, and cevimeline.

In still another alternative, the cholinomimetic is a cholinergic nicotinic receptor agonist. Typically, the cholinergic nicotinic receptor is selected from the group consisting of varenicline, galantamine, and nicotine.

In still another alternative, the cholinomimetic is sildenafil.

Another aspect of the present invention is directed to methods and compositions employing other therapeutic agents and combinations of therapeutic agents for emulating the theoretical pharmacological effects of the non-selective mAChR antagonist scopolamine.

Accordingly, one embodiment of this aspect of the present invention is a method for treatment of a mental or behavioral disorder comprising administration of a therapeutically effective quantity of an antagonist of muscarinic acetylcholine receptors of subtype M1 (M1 mAChR). Alternatively, the method can comprise administration of a therapeutically effective quantity of an antagonist of muscarinic acetylcholine receptors of subtype M2 (M2 mAChR), and/or administration of a therapeutically effective quantity of an antagonist of muscarinic acetylcholine receptors of subtype M4 (M4 mAChR).

Other alternatives for therapeutic agents in this aspect of the invention exist, including: (i) an agonist of M1 mAChR; (ii) an agonist of muscarinic acetylcholine receptors of subtype M2 mAChR; (iii) an agonist of muscarinic acetylcholine receptors of subtype M3 mAChR; (iv) an agonist of muscarinic acetylcholine receptors of subtype M5 mAChR; (v) both an antagonist and an agonist of M1 mAChR; (vi) a non-selective agonist of mAChR; (vii) an agent to increase acetylcholine (ACh) level (e.g., an acetylcholinesterase inhibitor); (viii) an antagonist of glutamate receptors; (ix) an agonist of nicotinic receptors; (x) an agonist of M2 mAChR and/or M4 mAChR to normalize ACh release; (xi) an agonist of opiate receptors to normalize ACh release; (xii) of an antagonist of CRF receptors to normalize ACh release; (xiii) an agent that antagonizes nitric oxide release; (xiv) an antagonist of nitric oxide receptors of subtype NR2B; (xv) an agonist of substance P receptors of subtype neurokinin 1 (NK1); (xvi) an antagonist of a cytokine or of a receptor of a cytokine; (xvii) a modulator of interferon receptors; (xviii) an agent that increases the level of P11 protein; (xix) an agonist of a serotonin 5HT1A receptor; (xx) an agonist of a serotonin 5HT1B receptor; (xxi) an agent that inhibits serotonin reuptake; (xxii) an agent that is a selective M1 antagonist; (xxiii) an agent that is a non-selective M1 antagonist; (xxiv) an agent that is an inverse agonist of the M1 receptor; (xxv) an agent that is a selective partial agonist of the M1 receptor; (xxvi) a non-selective partial agonist of the M1 receptor; (xxvii) a selective partial agonist of the M1 receptor and a non-selective partial agonist of the M1 receptor; (xxviii) a selective negative allosteric modulator of the M1 receptor; (xxix) a non-selective negative allosteric modulator of the M1 receptor; (xxx) a neutral allosteric modulator of the M1 receptor; (xxxi) a selective positive allosteric modulator of the M1 receptor; (xxxii) a non-selective orthosteric M1 antagonist and a non-selective negative allosteric modulator of the M1 receptor; (xxxiii) an orthosteric M2 antagonist, an allosteric modulator of the M2 receptor, and an inverse agonist of the M2 receptor; (xxxiv) an orthosteric M4 agonist; (xxxv) a negative allosteric modulator of the M4 receptor and a neutral allosteric modulator of the M4 receptor; (xxxvi) an orthosteric M2 full agonist; (xxxvii) an orthosteric M2 partial agonist; (xxxviii) a positive allosteric modulator of the M2 receptor; (xxxix) an orthostatic M4 full agonist, an orthosteric partial agonist of the M4 receptor, and a positive allosteric modulator of the M4 receptor; (xl) an orthostatic M3 full agonist, an orthostatic M3 partial agonist, and a positive allosteric modulator of the M3 receptor; (xli) an orthostatic M5 full agonist, an orthosteric partial agonist of the M5 receptor, and a positive allosteric modulator of the M5 receptor; (xlii) a benztropine compound or its analogs; (xliii) a diphenylpiperidine compound; (xliv) a mixed selective M1/M3 antagonist; (xlv) an antagonist of the metabotropic glutamate receptors (mGluRs) subtype mGluR1; (xlvi) an antagonist of the metabotropic glutamate receptors (mGluRs) subtype mGluR5; (xlvii) an antagonist of NMDA glutamate receptors; (xlviii) a selective antagonist of opiate receptor subtypes μ or δ; (xlix) an opiate receptor that is non-subtype-selective; or (l) an agonist of galanin receptor subtype 2 (GalR2).

These methods can further comprise the administration of a therapeutically effective quantity of a partial agonist of dopamine D2 receptors. In another alternative, methods as described above can further comprise the administration of a therapeutically effective quantity of an agent inhibiting dopamine reuptake. In still another alternative, methods as described above can further comprise the administration of a therapeutically effective quantity of an agent inhibiting norepinephrine reuptake. In still another alternative, methods as described above can further comprise the administration of a therapeutically effective quantity of an antagonist of norepinephrine α_{2C} receptors. In still another alternative, methods as described above can further comprise the administration of a therapeutically effective quantity of an antagonist of norepinephrine α_{2A} receptors. In still another alternative, methods as described above can further comprise the administration of a therapeutically effective quantity of an antagonist of norepinephrine α₂ receptors.

The methods as described above in these embodiments of this aspect of the invention can further comprise the administration of a therapeutically effective quantity of a compound from one of the following classes of psychotropic medications: antidepressants, antipsychotics, compounds demonstrating antipsychotic properties, mood stabilizers, stimulants, anxiolytics, hypnotics/sedatives, entheogens or nootropics (e.g., cognitive enhancer), anti-ADHD agents, antiaddictives, euphoriants, antidementia agents, depressants, anticonvulsants, analgesics, anesthetics (general, local), antimigraine agents, anorectics, antiparkinson's agents, neuroprotectives, orexigenics, or wakefulness-promoting agents.

Methods according to the present invention can be used to treat a broad range of mental and behavioral conditions.

The invention also encompasses pharmaceutical compositions. In general, a pharmaceutical composition according to the present invention comprises:

(1) a therapeutically effective quantity of one or more therapeutic agents affecting brain reward circuitry as described above; and

(2) a pharmaceutically acceptable carrier.

### BRIEF DESCRIPTION OF THE DRAWINGS

The following invention will become better understood with reference to the specification, appended claims, and accompanying drawings, where:

FIG. 1 is a diagram illustrating the brain reward circuitry.

FIG. 2 is a graph illustrating the effect of antagonism of M1 mAChR and agonism of mAChR in the nucleus accumbens shell (NAcShell). The discrete trial current-threshold intracranial self-stimulation paradigm was used to assess the effect of cholinergic drugs infused directly into the NAcShell on reward, independent of performance. See below and Markou and Koob (Neuropsychopharmacology. 1991 January; 4(1):17-26) for detailed description of the paradigm. Thresholds measured in a rat vary little in the 4 pre-drug days (<7% of the mean or baseline). The non-selective mAChR agonist arecoline dose-dependently elevates threshold (0.01 M, +11%; 1.0 M, +57.5%, N=2). Pirenzepine dihydrochloride (Sigma-Aldrich, Saint Louis, Mo.), a selective M1 mAChR antagonist, dose-dependently lowers threshold (0.1 mM, −7%; 100 mM, 14%, N=2). The drugs were infused into the NAcShell at a steady rate by reversed microdialysis over the hour required to complete threshold testing. The study clearly indicates that antagonism of M1 mAChR has a rewarding and mood elevating effect, and suggests that mAChR stimulation, including the M1 receptors, may do the opposite, by producing anhedonia.

FIG. 3 is a graph illustrating swimming time following injections of cholinergic drugs in NAc (black bars), compared to Ringer (white bars). (A) Local injection of arecoline, a muscarinic agonist, decreased swim time; the animals quickly gave up (40 μg, −41%, n=8, t=3.53, p<0.01; and 80 μg, −66%, n=6, t=4.12, p<0.01). (B) Pirenzepine, an M1 antagonist, increased swimming escape attempts in the manner of an antidepressant, but note this is a local injection (17.5 μg, n=8, +39%, t=3.63, p<0.01; 35 μg, +40%, n=9, t=7.15, p<0.001). (C) Local scopolamine, a mixed M1 and M2 antagonist, increased swim time (0.5 μg, n=10, N.S.; 1.0 μg, +50%, n=10, t=12.74, p<0.001). (D) Local gallamine, an M2 antagonist, decreased swim time (0.13 μg/side, −21%, n=7, t=3.72, p<0.01; 0.44 μg/side, −37%, n=8, t=2.95, p<0.05; 0.88 μg/side, −36%, n=7, t=4.64, p<0.01). Swimming time following Ringer is normalized to 100%, with each bar representing the mean of the normalized responses across subjects. Swimming time following a drug injection is expressed as a percentage of the swimming time following vehicle injection on a different day. Significant changes in the within-subject response to drug injections are indicated with asterisks (* p<0.05, ** p<0.01, *** p<0.001).

FIG. 4 is a graph illustrating the effects of cholinergic drugs on locomotor activity in a photocell cage. Activity in the 10 min following drug injection in the NAcShell (black bars) is expressed as a percentage of control injections normalized to 100% (white bars). The muscarinic agonist arecoline injected in NAcShell significantly decreased locomotor activity by reducing rearing and exploratory behavior (40 μg, n=6, * p<0.01). Responses to M1 and M2 antagonists were not significantly different from Ringer (pirenzepine, 35 μg, n=6, N.S.; scopolamine, 1 μg, n=4, N.S.; gallamine, 0.88 μg, n=7, p<0.08). These results indicate that pirenzepine and scopolamine do not act like a stimulant such as amphetamine.

FIG. 5 is a graph illustrating the effect of antagonism of mAChR and agonism of M2 mAChR in NAcShell on ACh efflux. To determine if the M2 drugs act presynaptically on autoreceptors controlling ACh release, freely moving rats received infusions of an M2 agonist or antagonist in the NAcShell for 20 min by reverse dialysis while extracellular ACh was measured. The M2 agonist, oxotremorine (4 mM perfusate inside the probe) decreased extracellular ACh to 55% of baseline in the hour following the infusion (n=4, F(9,27)=5.57, p<0.001). The M2 antagonist, gallamine at 1 mM probe concentration greatly increased ACh to a level 437% of baseline (n=5, F9,36=5.42, p<0.001). The effect was short lasting. ACh returned to baseline 20 min following the infusion. Scopolamine at 10 mM, to antagonize both M1 and M2 receptors, increased ACh 220% of baseline for more than 100 min (n=5, F9,36=9.23, p<0.001).

FIG. 6 is a graph illustrating illustrates the effects of local fluoxetine administration on ACh outflow in the NAcShell during the pretest and the swim tests. * p<0.05; † p<0.001, compared to Ringer. The ACh levels are expressed as percentages of baseline and not in pmole, because absolute levels were not recorded in this experiment. ACh levels during and following 1.0 mM fluoxetine infusion were substantially lower than levels recorded at similar time points during either Ringer infusion or during the pretest (p<0.05, two-way ANOVAs followed by Bonferroni post-hoc tests). Moreover, the swimming time during fluoxetine (329±45 sec) was 18.9% greater than during Ringer (277±44 sec, n=8; one-tail: t=1.89, p<0.005, two-tail: t=2.36, p<0.007). Two-way repeated measures ANOVA indicated significant effects of time (F(19,399)=22.70, p<0.0001) and treatment (F(2,399)=13.66, p<0.0001) on ACh outflow (% of baseline) in NAc, as well as a treatment by time interaction (F(38,399)=6.45, p<0.0001). On test Day 1, ACh decreased below baseline during the second half of the swimming session and remained suppressed during the next 30 min when the rats were removed from the water (p<0.05). On subsequent days (Day 2 or Day 3), during Ringer infusion, ACh again decreased half way into the swim test, but unlike the previous day, ACh remained suppressed for only 15 min after rats were removed from the water (p<0.05). On the days rats received the fluoxetine treatment, ACh dramatically decreased half way into the fluoxetine infusion and remained strongly suppressed below baseline during rest of the observation period (p<0.05). Between-group comparisons using Bonferroni's tests revealed that changes in extracellular ACh during the Ringer day and the pretest day were not different from each other.

FIG. 7 is a graph demonstrating the effects of chronic systemic fluoxetine administration on basal extracellular ACh in the NAcShell. The data suggest that basal ACh was elevated two weeks after the preswim in rats injected daily with saline (‡ p=0.06). The group treated daily with fluoxetine had a normalized basal ACh level. A Chi-square test indicated that chronic fluoxetine and chronic saline had opposing effects on basal ACh (p<0.0001, χ2 for independence). Daily subcutaneous injection of fluoxetine over 14 days counteracted the rise in basal extracellular ACh that otherwise followed a swim two weeks earlier. Basal extracellular ACh level tended to rise following a swim in the control group that received chronic, daily saline treatment (after pretest/before treatment: n=4, 1.52±0.3 pmol; after 14 days of treatment: 4.22±1.47 pmol; t=2.1, p=0.06). However, no change in basal extracellular ACh occurred follow chronic fluoxetine treatment (after pretest/before treatment: n=4, 2.80±0.9 pmol; after 14 days of treatment: 2.37±2.01 pmol, n.s.). A chi-square test further suggested that chronic fluoxetine and chronic saline treatment had opposing effects on the basal extracellular ACh levels, i.e., saline allowed it to rise, whereas fluoxetine kept it stable (p<0.0001, χ2 for independence). The total escape efforts, as indicated by the aggregate scores of swimming plus climbing, in the two treatment groups showed significant differences: the swimming plus climbing scores were higher following chronic fluoxetine treatment than following control chronic saline treatment (saline: 42±11; fluoxetine: 55±7; t=3.3, p<0.05). As expected, the immobility scores following chronic fluoxetine treatment (n=3, 65±7) showed the opposite trend; they were lower than the scores seen following chronic saline treatment (n=3, 77±11; t=3.3, p<0.05).

FIG. 8 is a schematic diagram illustrating the mechanisms of antidepressant action of scopolamine in the nucleus accumbens. In addition to blocking the M1 receptors [mechanism A], scopolamine also blocks M2 and M4 receptors on cholinergic interneurons to disinhibit ACh release [mechanism A]. Elevated ACh increases DA and GABA releases and decreases glutamate release to further alleviate depression and anxiety. ACh released by scopolamine stimulates M2 and M4 receptors on glutamate afferent terminals to inhibit glutamate release, thereby reducing NMDA receptor stimulation and activity in the medium spiny neurons (MSN) to further alleviate depression and anxiety [mechanism B]. ACh released by scopolamine stimulates M3 receptors on dopamine terminals to increase DA release [mechanism C]. ACh released by scopolamine also stimulates M4 receptors on the MSN projecting to the ventral tegmental area (VTA) to inhibit these MSN, resulting in disinhibition of DA release in the nucleus accumbens [mechanism D]. Scopolamine blocks M2 and M4 receptors on the cell bodies and terminals of cholinergic neurons projecting from the pedunculopontine tegmental nucleus to the VTA, increasing ACh release in the VTA. ACh released in the VTA stimulates M5 receptors located on DA neurons to release DA in the nucleus accumbens [mechanism E]. ACh released by scopolamine stimulates nicotinic receptors (non 7 subtype, especially the 6-containing subtype) on DA terminals to facilitate DA release [mechanism F]. Finally, ACh released by scopolamine stimulates nicotinic receptors on GABA interneurons and on the MSN GABA collaterals to release GABA, and increased GABA release in turn directly inhibits MSN, thereby further alleviating depression and anxiety [mechanism G].

### DETAILED DESCRIPTION OF THE INVENTION

Three recent human trials using scopolamine, an anticholinergic drug exhibiting the features of our invention, have demonstrated unprecedentedly rapid and long-lasting antidepressant and anti-anxiety effects in patients with major depression and bipolar disorder. All three studies showed that one single dose of scopolamine dramatically reduced depression and anxiety within 3-5 days (as opposed to the usual 3-4 weeks with currently available and widely used conventional antidepressants). Another extremely unusual feature of scopolamine is that the antidepressant/anti-anxiety effects from one single dose of scopolamine can last for more than 17 days. Moreover, the rates of remission from depression these human trials with scopolamine were about 4 times greater than rates seen with current antidepressants. Scopolamine was especially effective in women, as the rate of remission in female patients was reported to be as high as 71%. Scopolamine is able to ameliorate clinical symptoms even in patients who were resistant to all other conventional antidepressant medications.

According to the authors of these studies, “A promising aspect of the [studies] is the rapid onset of symptom relief observed with scopolamine treatment. One shortcoming of conventional antidepressant treatments is that the several-week delay needed to achieve clinically meaningful improvement prolongs patients' vulnerability to suicide and disability. Treatments that produce antidepressant responses within 1 week—electroconvulsive therapy, high-dose tricyclic antidepressant (TCA) drug administration, total sleep deprivation, and ketamine use—have not proved amenable to widespread clinical application because of their adverse effects or the transient nature of their therapeutic benefits. In contrast, the absence of serious adverse effects encountered in [these studies] suggests that scopolamine may provide a relatively safe and well-tolerated intervention for achieving rapid antidepressant responses.”

While scopolamine's antidepressant/anti-anxiety effects are clear, its mechanisms of action are not completely understood. It is essential to further clarify the mechanisms underlying the antidepressant/anti-anxiety action of scopolamine. Such drug mechanisms will uncover multiple novel drug targets for rapid and effective reversal of major depression and anxiety, as well as bipolar disorder.

Described herein are the Applicant's theorized biological mechanisms underlying the antidepressant action of scopolamine. Based on these mechanisms, the Applicant has formulated novel drug combinations that mimic the biological action of scopolamine, thereby rapidly reverse depression and anxiety. It is expected that such drug combination would have fewer side-effects than scopolamine, since each individual drug component has been commonly used clinically with minor, if any, side-effects.

The compositions and methods of the present invention for treating mental and behavioral disorders are based on modulation of the activity of muscarinic acetylcholine receptors (hereinafter “mAChR”), or modulation of the activity of nicotinic acetylcholine receptors (hereinafter “nAChR”), and/or normalizing the release of acetylcholine (hereinafter “ACh”) in the nucleus accumbens (hereinafter “NAc”).

Various alternatives of methods and compositions according to the present invention can involve: (1) antagonism of M1 mAChR; (2) agonism of M1 mAChR; (3) both antagonism and agonism of M1 mAChR; (4) non-selective antagonism of mAChRs; and (5) non-selective agonism of mAChRs.

In one alternative, the methods and compositions according to the present invention involve emulating the theoretical pharmacological effects of the non-selective mAChR antagonist scopolamine. As used herein, the term “emulating the theoretical pharmacological effects of the non-selective mAChR antagonist scopolamine” includes antagonism of M1 mAChR and one (or more) of the following: antagonism of M2 mAChR; antagonism of M4 mAChR; increasing ACh levels; agonism of M2 mAChR; agonism of M4 mAChR; antagonism of glutamate receptors; agonism of M3 mAChR; agonism of M5 mAChR; agonism of nAChRs; or agonism of GABA receptors.

Disclosed herein is a method for treating mental and behavioral disorders comprising normalizing ACh release, comprising one or more of the following: agonism of M2 mAChR; agonism of M4 mAChR; antagonism of glutamate receptors; agonism of opiate receptors; antagonism of CRF receptors; antagonism of nitric oxide release; antagonism of nitric oxide receptor subtype NR2B; agonism of substance P receptors (neurokinin 1); antagonism of cytokine or its receptor; modulation of interferon receptor; increasing the levels of P11 protein; agonism of serotonin 5HT1A receptors; agonism of serotonin 5HT1B receptors; and/or inhibiting serotonin reuptake.

Each of the above methods may further comprise partial agonism of DA D2 receptors.

Also disclosed herein is a method for treating mental and behavioral disorders comprising one or more of the above described methods and one (or more) of the following: Inhibiting dopamine reuptake; Inhibiting norepinephrine reuptake; antagonism of norepinephrine alpha-2C receptors; agonism of norepinephrine alpha-2A receptors; antagonism of norepinephrine alpha-2 receptors; and/or agonism of norepinephrine alpha-2 receptors.

Also, the above methods provide utility when combined with other medications by affecting the pharmaceutical properties of those other medications.

This application provides novel methods for treating a variety of mental and behavioral disorders. Those mental and behavioral disorders can be recognized by identifying the clinical symptoms and/or syndromes of mental and behavioral disorders described in standard diagnostic manuals (e.g., DSM-IV-IR, ICD-10); specific clinical symptoms shared by different diagnoses of mental and behavioral disorders described in standard diagnostic manuals; and subclinical symptoms or complaints regarding mental and behavioral disorders decreasing a person's quality of life or preventing a person from functioning optimally.

This application provides a method for normalizing, decreasing, or enhancing neurotransmission in the brain reward circuitry, thus enabling the treatment of depression and other mental and behavioral conditions as described further below. As used herein, the term “brain reward circuitry” comprises interconnecting brain regions including the lateral hypothalamus (LH), medial forebrain bundle (MFB), ventral tegmental area containing dopamine neurons (VTA), the nucleus accumbens shell or nucleus accumbens medial core, ventral pallidum, orbitofronal cortex (OPFC), medial frontal cortex (MPFC), cingulate cortex, lateral bed nucleus of the stria terminalis of the amygdala (BNST), mediodorsal thalamus (mdTham), lateral septum, the dorsal raphe containing serotonin neurons, the locus coeruleus containing norepinephrine neurons.

The term “brain reward circuitry” was first conceived from the observation that rats voluntarily and rigorously perform operant tasks to receive electrical or chemical stimulation in areas within this circuitry noted above, a phenomenon referred to as “intracranial self-stimulation (ICSS)” or “brain reward stimulation (BRS). The brain reward circuitry mediates reward and incentive motivation for natural reward, as well as unnatural rewards such as drugs of abuse. Without being bound by any theory, the applicant hypothesizes that this brain network in both humans and animals mediates a broad-spectrum of emotion/emotional expression (from pleasure to aversion) and behavior (including instinctual, habitual, and complex goal-oriented behaviors). Accordingly, Applicant has developed methods for normalizing, decreasing, or enhancing neurotransmission in the brain reward circuitry. These methods provide broadly applicable benefits to treating a wide variety of diseases and conditions.

In one embodiment, this disclosure provides a method of normalizing, decreasing, or enhancing neurotransmission in subregions of the ventral striatum.

In another embodiment, this disclosure provides a method of normalizing or enhancing neurotransmission in the nucleus accumbens. Without being bound by any theory, the applicant hypothesizes that the nucleus accumbens region of the brain is the central component of the brain reward circuitry.

The ventral striatum consists of the nucleus accumbens, the olfactory tubercle, and the ventromedial parts of the caudate nucleus and putamen. It is strongly innervated by dopaminergic fibers from the ventral tegmental area (VTA), known as the mesolimbic dopamine system, and has the highest density of serotonergic inputs in the striatum. The ventral striatum accommodates massive projections from corticolimbic structures (including the prefrontal cortex, amygdala, hippocampus, thalamus) encoding cognitive, emotional, and sensory information, as well as inputs from subcortical structure (e.g., the hypothalamus) and circulating hormones, neuropeptides, and other factors (including those related to the immune system) encoding information regarding the internal state of the body. The ventral striatum integrates all such information to guide behavior and modulate affect.

In one embodiment, the methods disclosed herein comprise normalizing neurotransmission in the medial subregion of the NAc (hereinafter “the NAcShell”), which is a phylogenetically old structure existing in both humans and in lower species. The NAcShell in humans receives inputs selectively from limbic structures (e.g., the ventromedial prefrontal cortex, hippocampus, and amygdala), as opposed to other brain structures involved in motor control. Without being bound by any theory, the applicant hypothesizes that the NAcShell plays a crucial role in the mediation of basic instinctual emotions along the spectrum from reward to aversion that serve as conscious cues to guide the planning and execution of complex behaviors appropriate to the changing environment.

The NAcShell also makes the final decision whether to initiate approach behavioral (e.g., exploratory, reward seeking) or avoidance behavior (active escape from or passive coping with an aversive situation). Through its discrete output pathways (i.e., the axonal projections from the so called “GABA output neurons” also referred to as “medium spiny neurons”), the NAcShell can increase (or decrease) activities in the cortical and thalamic brain circuitry involved in environmental awareness or “externalization” and action planning and execution, which interact (often in a reciprocal manner) with brain circuits for “internalization” or self-awareness. The NAcShell is involved in the mediation of incentive motivation and in impulse control through its inhibitory feedback projection to dopamine (DA) neurons in the midbrain's ventral tegmental area that sends dopaminergic projections to the corticolimbic structures (the mesocortical DA pathway) and dopaminergic projections to nuclei in the basal forebrain, including NAcShell and other parts of the ventral striatum (the mesolimbic DA pathway). Without being bound by any theory, the applicant hypothesizes that the NAcShell also influences arousal, feeding, autonomic regulation, and nociception through its projections to the hypothalamus and the nuclei in the brain stem.

Accordingly, without being bound by any theory, Applicant hypothesizes that normal neurotransmission in the NAcShell is required for an individual to maintain conventionally acceptable experience and expression of emotion and for adaptive behavior. Abnormal neurotransmission in the NAcShell has been increasingly implicated in a variety of mental and behavioral disorders, including depression, anxiety, PTSD, substance use disorder, schizophrenia, eating disorders, obsessive-compulsive and anxiety disorders, attention deficit hyperactivity, sleep disorders, decreased pleasure and motivation, and substance (e.g., neuroleptic) induced dysphoria.

The methods herein provide benefits in treating one or more mental and behavioral disorders; to treat specific clinical symptoms shared by different primary diagnoses. The methods herein provide benefits in treating mental and behavioral conditions considered subclinical. The methods herein provide benefits in treating and enhancing a person's overall functioning.

Disclosed herein are methods of agonizing and/or antagonizing one or more subtypes of muscarinic acetylcholine receptors (mAChR), agonizing one or more subtypes of nicotinic acetylcholine receptors (nAChR), and normalizing the release of acetylcholine (ACh) in the NAcShell.

In one embodiment of the invention, the disclosed cholinergic methods are combined with additional methods, which include agonism and/or antagonism of subtypes of the receptors of glutamate, γ-aminobutyric acid (GABA), serotonin, norepinphrine, and other factors. Combining these two methods provides a method that further normalizes neurotransmission in the NAcShell.

In one embodiment, the methods disclosed herein comprise administering one or more drugs, each of which exhibits one feature of the invention (i.e., single-action drug).

In one embodiment, the methods disclosed herein comprises administering one or more drugs, each of which exhibits of more than one features of the invention (i.e., multi-action drugs).

In one embodiment, the methods disclosed herein comprise administering a multi-action drug with a single-action drug to complete the formulation of the invention. In one application, a combination of one or more single-action drug and/or one or more multi-action drug is used as an adjunct treatment to improve the efficacy of an existing medication (e.g., a medication is missing the essential feature of the invention exhibited by the adjunctive drugs). Such approach is expected to be readily implemented and cost-effective.

In one embodiment, the methods disclosed herein comprise administering one or more single- or multi-action drugs. In one embodiment, these drugs are chosen from (a) medication indicated for the conditions in which the method applies; (b) medications indicated for other conditions; (c) medications known to be relatively safe, but which have not demonstrated efficacy in clinical trials for other conditions; (d) experimental drugs that have been used experimentally in animals but have not been tried in humans; (e) existing compounds or analogs of certain class of compounds that have not been used in humans or used experimentally in animals. All of the above drugs can be identified in drug databases as well as in the literature.

Applicant was first to identify the NAcShell as scopolamine's therapeutic target (Chau et al., Ann N Y Acad. Sci. 1999 Jun. 29; 877:769-74; Chau et al., Neuroscience. 2001; 104(3):791-8; Chau's Ph.D. Thesis 2001). Scopolamine was later shown to be extremely rapidly acting and highly efficacious antidepressant in human trials (Furey and Drevets, Arch Gen Psychiatry. 2006 October; 63(10):1121-9; Drevets and Furey, Biol Psychiatry. 2010 Mar. 1; 67(5):432-8). However, because of its broad-spectrum pharmacological profile resulting in potentially some side-effects, scopolamine is currently not indicated for use beyond treatment of certain somatic disorders such as motion sickness. The mechanism through which scopolamine rapidly reverses depression and anxiety is unknown. The current invention includes a theorized mechanism of action of scopolamine that occurs in the NAcShell. The methods of the invention derived from this theorized mechanism are expected to exhibit equal if not greater efficacy than scopolamine and to not exhibit the side effects associated with scopolamine.

Without being bound by any theory, Applicant hypothesizes that that depression and anxiety is mediated by elevated basal ACh level in the NAcShell and that antidepressants/antianxiety drugs act by lowering or normalizing the elevated basal ACh in NAcShell. Accordingly, the invention comprises methods for treating depression and anxiety by administering pharmacological agents that have been demonstrated or predicted to lower ACh release in the NAcShell and/or agents that normalize the function of the cholinergic interneurons in the NAcShell that release ACh. The site of action of the methods of the current invention is not limited to the NAcShell and may include other brain regions involved in the conditions wherein the methods apply.

Normal affect (especially reward and hedonic capacity) and motivation allow a person to adapt and thrive in various environments. A number of mental and behavioral disorders, including depression, anxiety, PTSD, schizophrenia, and substance use disorder exhibit severe deficits (or excessive increase) in reward and motivation, abnormal decrease or increase in the range of emotion (including hedonic capacity) and range of motivation, or negative allostatic shift in reward and incentive motivation away from equilibrium (e.g., decreased pleasure from and motivation to engage in everyday activities due to drug abuse).

Examples of such polar opposite affects and motivation as described in standard diagnostic manuals (e.g., DSM-IV-IR or ICD-10) include dysphoria or aversion vs. sense of relief, anhedonia vs. pleasure, flat affect vs. labile emotion or mania, depressed mood vs. satisfaction or happiness, anergia and psychomotor retardation vs. hyperactivity and mania. In patients with bipolar disorders, mood and motivation fluctuate between periods of hypomanic symptoms and numerous periods of depressive symptoms. In schizophrenia, however, extreme affective and motivational symptoms may occur at various stages of the disorder. Disorder of affect and motivation is evident during a psychotic episode, in which a patient may exhibit a distorted sense of personal efficacy (delusion of grandeur), or profound immobility or stupor (catatonia). Chronic negative symptoms occur in between psychotic episodes and often worsen over time. Such negative symptoms include restriction in the range and intensity of emotional expression (affective flattening), in the fluency and productivity of thought and speech (alogia), in the initiation of goal-directed behavior (avolition or decreased drive), and in the ability to relate to other (e.g., behavioral or social withdrawal).

Marked decrease in hedonic capacity (anhedonia) is increasingly recognized as a pervasive symptom of schizophrenia responsible for the high rates of substance abuse among patients with schizophrenia associated with dramatically worsening clinical course and high rates of suicide. The symptoms of depressive disorder are more clear cut; they include anhedonia (expressed by patients in various ways such as feeling “emotionally empty”) or diminished pleasure, persistent depressed mood or feelings of profound sadness (sometimes expressed by patients as “psychic pain”), irritability, diminished motivation or drive, persistent lethargy or feelings of decreased energy, and psychomotor retardation.

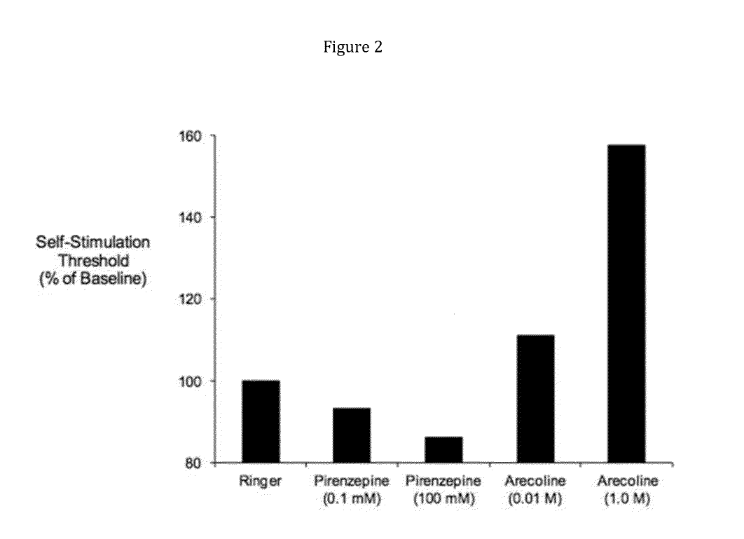

Surprisingly, Applicant discovered that antagonism of M1 mAChR in the NAcShell increases reward sensitivity in rats, as assessed using the discrete trial current-threshold discrete-trial self-stimulation (ICSS) procedure (see below for a description of this procedure). Antagonism of M1 mAChR was implemented by continuously infusing a selective competitive orthosteric antagonist of M1 mAChR (pirenzepine) directly into the NAcShell during the ICSS procedure. Infusing pirenzepine into the NAcSh dose-dependently lowers ICSS threshold (FIG. 2).

Applicant has also developed a method of non-selective agonism of mAChRs in the NAcShell decreasing reward sensitivity in rats. Infusing a non-selective full agonist of mAChRs (arecoline) into the NAcShell dose-dependently increases ICSS threshold (see FIG. 2).

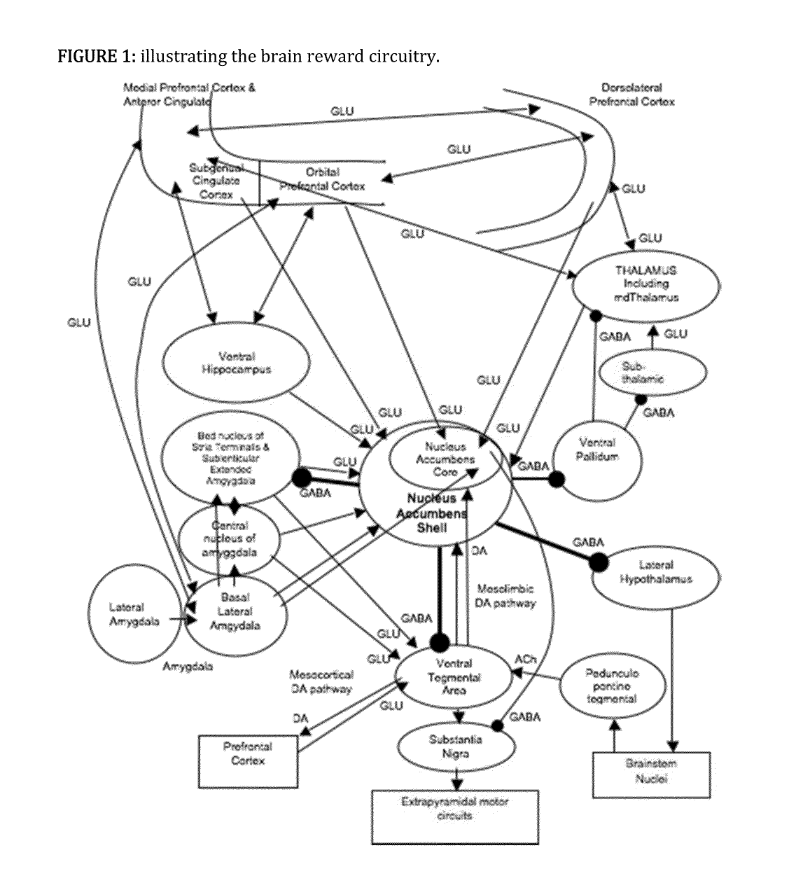

Anhedonia may be assessed by applying the intracranial self-stimulation (ICSS) paradigm. The ICSS may be used to assess anhedonia, reward, hedonic capacity, and motivation in animals. In this paradigm, a rat performs an operant task (e.g. pressing a bar) to receive rewarding electrical stimulation through electrodes implanted into specific areas of the brain, including the lateral hypothalamus (LH), medial forebrain bundle (MFB), ventral tegmental area containing dopamine neurons (VTA), the nucleus accumbens shell or nucleus accumbens medial core, ventral pallidum, orbitofronal cortex (OPFC), medial frontal cortex (MPFC), cingulate cortex, lateral bed nucleus of the stria terminalis of the amygdala (BNST), mediodorsal thalamus (mdTham), lateral septum, the dorsal raphe containing serotonin neurons, the locus coeruleus containing norepinephrine neurons (see FIG. 1). As noted above, these regions supporting ICSS form an interconnected network commonly referred to as the “brain reward circuitry.”

The most sensitive areas in which the lowest electrical intensity is required to elicit an operant response is the lateral hypothalamus, the nucleus accumbens, and sites along the medial forebrain bundle (the axonal tract carrying the axonal projections of dopamine neurons from the VTA). Stimulating these sites activates other brain areas supporting ICSS, including the mesolimbic DA pathway that releases DA in the NAc. These sites mediate natural reward and incentive motivation, because stimulation administered in such areas elicits natural reward-seeking and reward-consumatory behavior. For example, stimulating the lateral hypothalamus elicits eating and sexual activity in animals and stimulating the nucleus accumbens elicits feelings of pleasure in humans.

A skilled artisan would recognize that variations of the ICSS paradigm may be used. For example, one of the most commonly used ICSS procedures, which has been experimentally validated and has shown to be reward-selective, is the discrete-trial current-intensity threshold procedure. This procedure directly and reliably assesses an animal's sensitivity to rewards, hedonic capacity, and motivation, independent of performance, i.e., ability to move (for detailed procedures see Markou and Koob, Neuropsychopharmacology. 1991 January; 4(1):17-26). The discrete trial current-intensity threshold ICSS procedure provides unique ways to investigate the anatomical and neurochemical basis of reward and motivation and is an important tool for assessing of the reward-facilitating or anhedonic effects of various drugs or medications.

Lowering of ICSS thresholds indicates facilitation of brain stimulation reward, whereas elevation in ICSS threshold indicates diminished reward value of the stimulation and thus an anhedonic state. Acute administration of most drugs of abuse, including cocaine, amphetamine, nicotine, morphine, and heroin, lower ICSS thresholds in experimental animals, logically because of their rewarding values. By contrast, withdrawal from chronic administration of these drugs elevates ICSS thresholds, indicating an anhedonic state that resembles the negative affective and motivational state of the drug withdrawal syndrome experienced by humans. Elevated ICSS threshold also occurs in animal models of depression. Such elevation in ICSS threshold reflects decreased sensitivity to rewarding stimuli, decreased hedonic capacity or motivation, and/or an aversive and dysphoric affective state of the animal model of depression. Importantly, administration of antidepressants normalizes or lowers ICSS threshold in such animals, indicating a reversal of reward and motivational deficits.

The Porsolt swim test is an animal model of depression widely used as a screen for drugs with potential antidepressant effects in humans and for elucidating the pharmacological mechanisms of action of antidepressant medications. For example, the following procedure may be used:

On the first day of this test (day 1), a rodent (typically a rat) is placed in a cylindrical water tank for 15 min. Then after 24 hours (day 2) and 48 hours (day 3), drug and Ringer are administered to the animal in counterbalanced order. Immediately following each injection, the animal is placed in the swim test for 10 min. The length of time it swims during the swim tests is recorded. “Swimming” is defined by escape behaviors (i.e. diving, rigorous paddling with all four legs, circling the tank, and clambering at the tank walls). ‘Immobility’ was scored as floating and treading water just enough to keep the nose above water. Typically, after the first swimming session, animals become behaviorally depressed as shown by swimming less on later test days (day 2 or 3). The swim test has a high predictive validity in detecting antidepressants, as immobility in the swim test produces the highest correlation (r(s)=0.58) with a drug's clinical potency, relative to other tests (Willner, Psychopharmacology (Berl). 1984; 83(1):1-16). Antidepressants increase swimming without affecting locomotor activity, whereas non-antidepressant drugs such as certain psychostimulants may also decrease immobility, but also increase locomotor activity in the open field test. Therefore, to screen a potential antidepressant, the swim test is typically followed by test for hyperactivity in the open field.

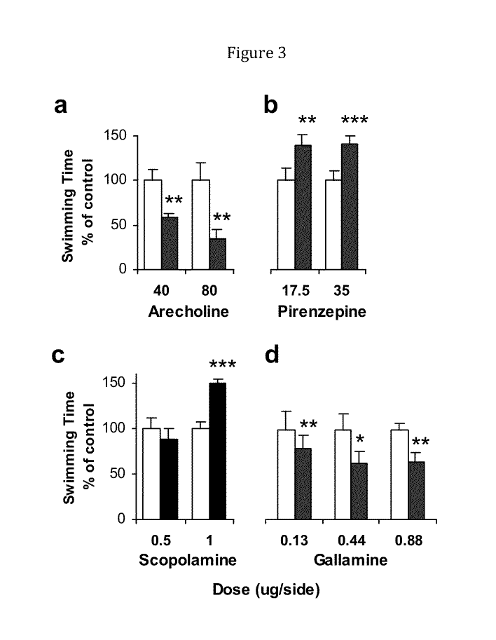

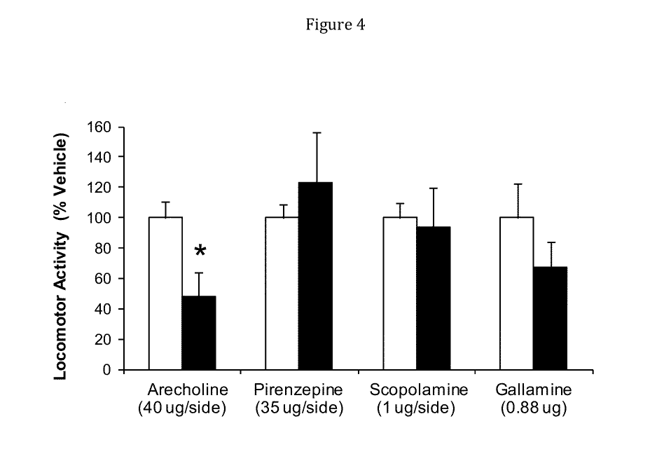

In one example, the inventor administered agonists and antagonists of mAChR administered into the NAcShell and assessed their potential antidepressant effects (or depression-inducing effects) using the above-described Porsolt swim test (Chau et al., Neuroscience. 2001; 104(3):791-8). Infusing pirenzepine into the NAcShell decreases immobility in the swim test (Table 1 and FIG. 3) without altering activity in the open field (FIG. 4). Infusion of scopolamine into NAcShell also decreases immobility in the swim test, presumably in part by blocking the M1 mAChR without altering activity in the open field (see Table 1 and FIGS. 3 and 4). Pirenzepine is a selective M1 mAChR orthostatic antagonist. Scopolamine is a non-selective competitive orthosteric mAChR antagonist exhibiting similar affinities for M1-M5 receptors.

Selective serotonin reuptake inhibitors (SSRIs) belong to one of the most commonly used classes of antidepressants. Accordingly, Applicant recognized that it was important to investigate how they may affect cholinergic transmission in NAcShell during depression (Chau et al., Neuropsychopharmacology. 2011 July; 36(8)1729-37). Applicant has determined that cholinergic interneurons in the NAcShell are important therapeutic targets of the antidepressant drug fluoxetine, a selective serotonin reuptake inhibitor (SSRI). Also, Applicant has assessed the effects of fluoxetine infusion into the NAcShell and the effects of chronic, daily subcutaneous injection of fluoxetine on extracellular ACh levels in the NAcShell in conjunction with behavioral measurements of escape motivation during the Porsolt swim test. The major results were as follows:

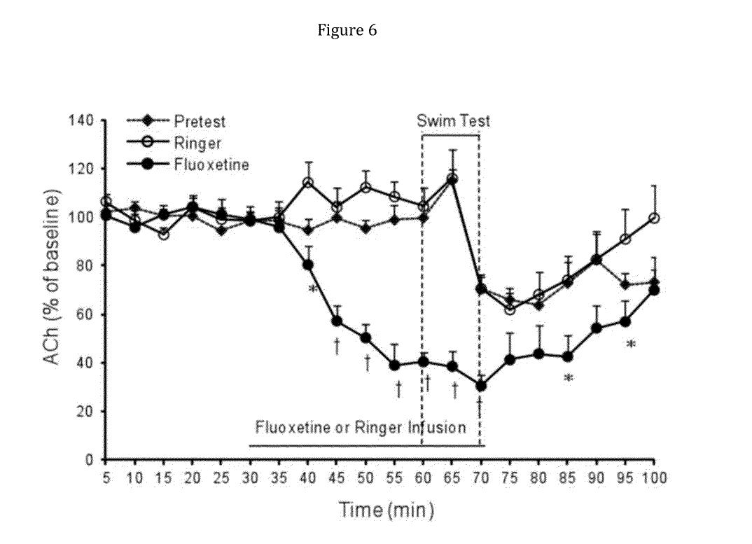

Infusing fluoxetine unilaterally into NAcShell decreases the extracellular level of ACh and simultaneously decreases immobility in the swim test (FIG. 6).

Infusing fluoxetine bilaterally into NAcShell dose-dependently and decreases immobility in the swim test, but does not affect locomotor activity in the open field (Tables 2-4).

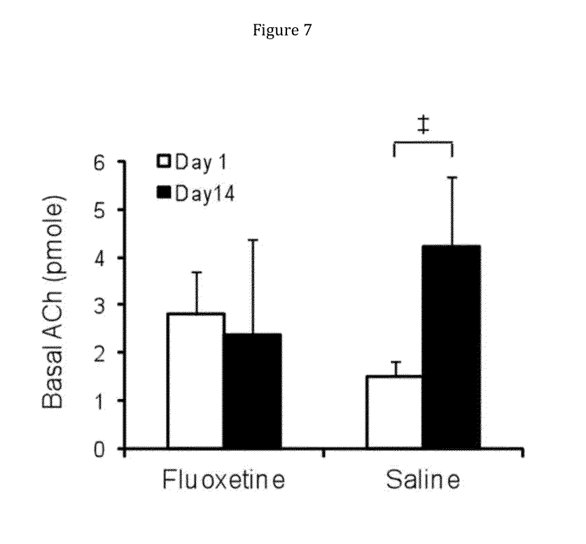

Injecting a therapeutically relevant low dose of fluoxetine subcutaneously, daily over 14 days normalizes the chronic elevation in basal ACh level occurring in the NAcShell during the behaviorally depressed state (in rats undergoing the Porsolt's forced swimming test) and decreases immobility in the swim test (FIG. 7).

One important aspect of this invention is that fluoxetine infused directly into the NAcShell alleviates signs of depression and decreases extracellular ACh level in an animal model of depression, instead of a non-depressed rat (see FIGS. 6 and 7).

Another important aspect of this invention is that the basal level of ACh in NAcShell remains elevated for more than 14 days following the initial swim, suggesting that chronic elevation in ACh in NAcShell may be an important factor contributing to depression. This hypothesis is supported by electrophysiological studies demonstrating that elevating the basal level of ACh in the NAc prevents the output medium spiny neurons (MSN) from exhibiting long-term depression (LTD). LTD occurs in the MSN when these neurons are repetitively stimulated. However, if basal ACh in the NAcShell is chronically elevated during depression, as suggested by the current data, it will prevent MSN from exhibiting LTD when repeatedly being stimulated by increased glutamate inputs from the limbic cortex (especially the subgenual cingulate cortex) and thalamus, both of which have been shown to be hyperactive during depression in humans. Consequently, ACh-induced inhibition of LTD in MSN is expected to cause MSN to become chronically hyperactive. Because MSN hyperactivity is thought to underlie decreased reward and motivation, anhedonia, aversion, and dysphoria during depression and drug withdrawal, antidepressant may alleviate these symptoms by lowering basal ACh. The current data indicating that fluoxetine treatment lowers basal ACh in NAcShell while simultaneously alleviating signs of depression provides direct evidence supporting this line of reasoning.

Thus, the Applicant has determined a new mechanism for alleviating depression involving lowering basal ACh in the NAcShell. Accordingly, disclosed herein are methods for treating depression, related disorders, or other disorders comprising ways to normalizing basal ACh in NAcShell.

Applicant has further elaborated on the mechanism of action of fluoxetine as follows: (A) Systemic daily administration of therapeutically relevant amounts of fluoxetine takes several weeks to produce an antidepressant effect, because such fluoxetine treatment takes that much time to lower or normalize the elevation in ACh levels in the NAc. (B) Fluoxetine infused directly into the NAc produces rapid antidepressant effects in a manner analogous to the action of the M1 antagonists administered systemically or locally in the NAc. (C) The long-lasting therapeutic effect of locally administered fluoxetine (or chronic systemic administration of fluoxetine) is mediated in part by one or more of the following: the presence of therapeutically effective amount of 5HT in the NAc; the presence of therapeutically effective amount of the metabolites of 5HT in NAc; the presence of therapeutically effective amounts of serotonin and/or its metabolites triggers certain therapeutic molecular or cellular processes that persist even after the surge in 5HT and its metabolites in the NAc have dissipated following acute fluoxetine administration. (D) Fluoxetine may also produce its antidepressant effects in part by elevating (or normalizing) DA levels in the NAc. (E) Finally, the Applicant integrated his past data with the data reported in this recent publication (Chau et al., Neuropsychopharmacology. 2011 July; 36(8):1729-37) and proposed: fluoxetine alleviates behavioral depression in part through the following mechanisms. First, fluoxetine treatment elevates extracellular 5-HT in the NAc by blocking its reuptake. Second, elevated 5-HT, through its stimulation of the 5-HT1A receptors, decreases or normalizes the elevation in ACh outflow from the local cholinergic interneurons during the behavioral depressed state. Finally, decreased ACh outflow in turn decreases cholinergic M1 receptor stimulation, thereby alleviating behavioral depression. Such intrinsic serotonergic and cholinergic mechanisms could potentially be one of the important targets of SSRI (and possibly other) treatments.

Disclosed herein are methods of treating the symptoms of various mental and behavioral disorders comprising antagonism of M1 mAChR. Exemplified mental and behavioral disorders in which the method applies include abnormalities in reward and motivation characteristic of depressive disorders, bipolar Disorders, schizophrenia, and substance use disorder, as noted above. Antagonism of M1 mAChR is also useful for treating abnormalities in reward and motivation occurring in (and possibly underlying the etiology of) other disorders, including neurodevelopmental disorders, anxiety disorders, sexual and gender Identity disorder, eating disorder, impulse control disorders, personality disorder, and sleep disorders. Antagonism of M1 mAChR in the NAcShell may be most effective in normalizing abnormalities in reward and motivation occurring in various mental and behavioral disorders. This is extremely plausible, given the fact that the NAcShell has been implicated in virtually all of the disorders noted above.

Disclosed herein are methods of treating conditions opposite to the symptoms of depressive disorders such as excessive and abnormal elation or pleasure, mania, hyperactivity, behavioral disinhibition, or abnormal incentive motivation comprising administering non-selective mAChR agonists or selective M1 mAChR agonists. Such conditions occur in a variety of mental and behavioral disorders, including certain neurodevelopmental disorders, bipolar disorders (e.g., mania), schizophrenia (e.g., psychosis), substance-related disorder (e.g., abnormal incentive motivation for drugs of abuse), over eating, impulse control disorders, hypersexual activity disorders, and certain personality disorders.

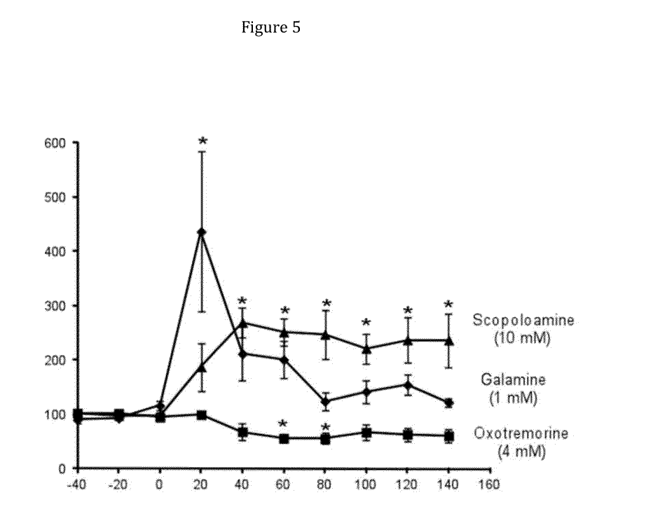

Also disclosed herein are methods of increasing the activities of M2 autoreceptors located on cholinergic interneurons to increase ACh release comprising administering gallamine. In one embodiment, administering gallamine elevates ACh levels (FIG. 5) and, in turn, increases the activities of postsynaptic M1 mAChR to exacerbate immobility in the swim test (Table 1). In another embodiment, antagonism of M2 mAChR and partial agonism of M1 mAChR using drugs with a dual action exhibiting both mechanisms increases ACh release throughout the brain while stabilizing activity at the M1 AChR and may be beneficial for treating certain conditions such as cognitive impairment and preventing depression in Alzheimer's disease.

As noted above, the methods of antagonizing M2 mAChR provide methods of treating conditions such as cognitive impairment in Alzheimer's diseases in which ACh release is deficient. However, elevating basal ACh release in the NAcShell may lead to hyperstimulation of local M1 mAChR, leading to depression and anxiety. Non-selective mAChR antagonists are beneficial for treating certain conditions, including depression, as exemplified by the effects of scopolamine in depressed patients. The current invention includes the theoretical mechanisms underlying the antidepressant effect of scopolamine and possibly of other non-selective antagonists of mAChRs (see below of a detailed description of these theoretical mechanisms). These mechanisms serve as a basis for methods according to the present invention, including methods employing therapeutic agents or combinations of therapeutic agents as described further below.

Applicant has elucidated various aspects concerning the mechanism of action for the drug scopolamine. This disclosure draws on this contribution and provides various methods of treatment.

Data regarding pirenzepine decreasing immobility in the swim test in a non-dose-dependent manner suggest that therapeutic effect of doses of pirenzepine used reached a ceiling effect (see FIG. 3 and Table 1).

Data regarding scopolamine decreasing immobility more than pirenzepine suggest that beside antagonism of M1 mAChR, antagonism of other mAChR subtypes may contribute to the overall antidepressant effect of scopolamine (see FIG. 3 and Table 1).

Disclosed herein is a method of administering scopolamine to potently decrease immobility in the swim test and to rapidly and effectively alleviate depression and anxiety in human trials is due to a combination of actions, including its ability to increase ACh release (see FIG. 5) together with its blockade of M1 mAChR.

This application includes additional mechanisms (and methods) regarding how scopolamine-induced increase in ACh release may contribute to its antidepressant/antianxiety effects.

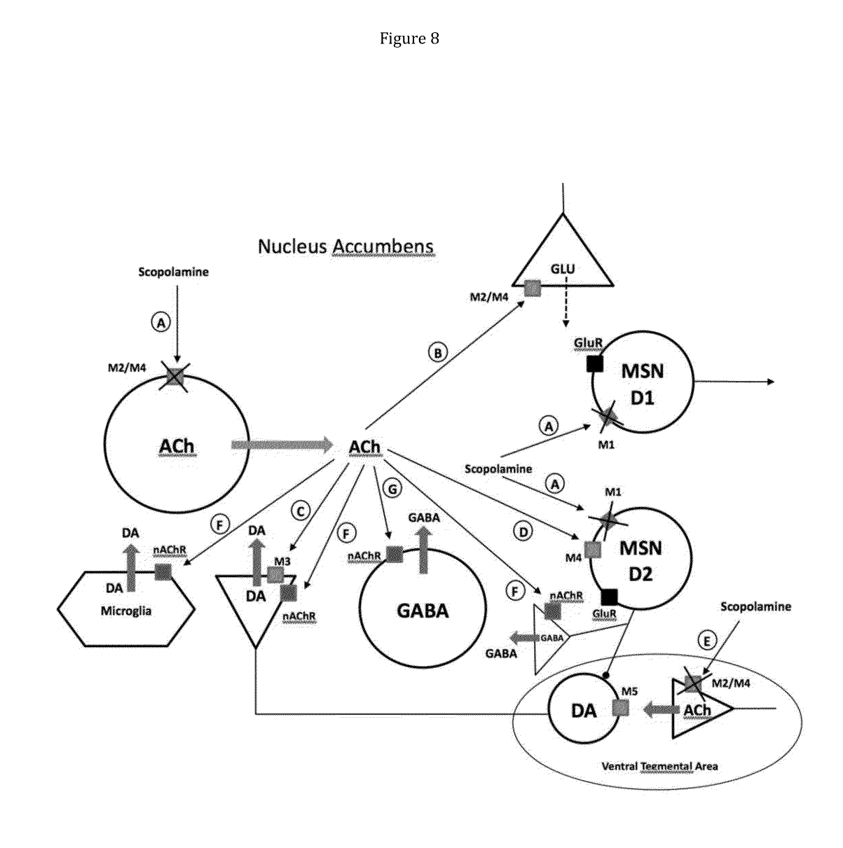

The overall mechanisms (denoted Mechanisms A-G, below), providing additional methods for treating depression and anxiety comprising using scopolamine, are described below. See also FIG. 8.

Mechanism A of Scopolamine

M1 receptors are located on the medium spiny neurons (MSN) that projects out of the NAc to other brain regions involved in the regulation of affect and behavior. M2/M4 autoreceptors in the NAc located on the local cholinergic interneurons help maintain ACh release within normal limits. M2/M4 receptors are less sensitive than postsynaptic M1/M3/M5 excitatory receptors; therefore M2 and M4 receptors are activated when extracellular ACh level is high, resulting in decreased ACh synthesis and release. The inventor's data showed that scopolamine (a non-selective antagonist of multiple mAChR subtypes, including M2 and M4 receptors) elevates ACh level in the NAc, whereas oxotremorine (a M2/M4 agonist) does the opposite by decreasing ACh (Chau et al., Neuroscience. 2001; 104(3):791-8).

Without being bound by any theory, the applicant hypothesizes that in addition to blocking the M1 receptors, scopolamine also blocks M2 and/or M4 receptors to release ACh, and such ACh release in turn stimulates one or more of postsynaptic mAChR subtypes (M2, M3, M4, and M5 receptors) and nicotinic acetylcholine receptors (nAChR) to further alleviate depression and anxiety.

Accordingly, the invention comprises antagonism of M1 mAChR (to ensure the M1 receptor pathway mediating the depression and anxiety is blocked) and one or more of the followings: antagonism of M2 mAChR, antagonism of M4 mAChR, and increasing ACh levels.

Scopolamine is a competitive antagonist at musarinic receptors. Its binding affinities for M1 and M3 receptors are approximately one order of magnitude higher than those of M2 and M4 receptors [M1 (Ki=0.085)≈M3 (Ki=0.063)>>M2 (Ki=0.88)≈M4 (Ki=0.1)] (Billard et al. J Pharmacol Exp Ther. 1995 April; 273(1):273-9). Therefore, higher doses of scopolamine are required to inhibit M2/M4 receptors than the doses required to inhibit M1-M5 receptors. Thus, higher doses of scopolamine are required to block M2/M4 than M1-M5. Applicant's data show that a sufficiently high dose of scopolamine elevates ACh level in the NAc to alleviate depression, while a low dose slightly exacerbates depression (Chau et al., Neuroscience. 2001; 104(3):791-8). Our data are consistent with the fact that low dose scopolamine used in early human trials yielded negative results, whereas higher dose scopolamine used in recent trials produces dramatic antidepressant effects in depressed humans (Furey and Drevets, Arch Gen Psychiatry. 2006 October; 63(10):1121-9; Drevets and Furey, Biol Psychiatry. 2010 Mar. 1; 67(5):432-8).

Mechanism B of Scopolamine

In the NAc, M2/M4 receptors are also located on glutamate-containing axonal terminals that originate from neurons located in corticolimbic and thalamic regions involved in depression and anxiety. The Applicant's data showed that (1) glutamate is released in the NAc during depression and the anxious state, and (2) glutamatergic NMDA receptor antagonists (dizocilpine, AP-5) alleviate depression when injected into the NAc (Rada et al., 2003, Neuroscience. 2003; 119(2):557-65). These data are consistent with several human trials indicating that ketamine, a NMDA receptor antagonist, has a rapid antidepressant effect (Zarate et al., Am J. Psychiatry. 2006 January; 163(1):153-5).

Without being bound by any theory, the applicant hypothesizes that ACh released by scopolamine stimulates postsynaptic M2/M4 receptors to inhibit glutamate release, thereby lessening NMDA receptor stimulation to further alleviate depression and anxiety.

Accordingly, the mechanism of one aspect of the present invention comprises one or more of the following: agonism of M2 mAChR, agonism of M4 mAChR, and antagonism of glutamate receptors.

Mechanism C of Scopolamine

In the NAc, M3 receptors are located on DA terminals. Stimulating M3 receptors is known to facilitate DA release release in the NAc.

Without being bound by any theory, the applicant hypothesizes that ACh released by scopolamine stimulates M3 receptors to elevate DA level in the NAc, thereby further alleviating depression and anxiety. One may argue that scopolamine itself blocks M3 receptors, and this action may counteract the beneficial effects of scopolamine-induced ACh acting on the same M3 receptors. However, Applicant theorizes that the large phasic release of ACh by scopolamine will be sufficient to displace smaller amounts of scopolamine occupying the M3 receptors. Thus, we expect an antidepressant net effect at the M3 receptors.

Accordingly, the mechanism of one aspect of the present invention comprises agonism of M3 mAChR.

Mechanism D of Scopolamine

Although some M4 receptors in the NAc are present on cholinergic interneurons, most of these receptors are located on the MSN. Stimulating these postsynaptic M4 receptors inhibits MSN, which then disinhibit DA neurons in the VTA to promote DA release in the NAc.

Without being bound by any theory, the applicant hypothesizes that ACh released by scopolamine stimulates M4 receptors to promote DA release, thereby further alleviating depression and anxiety.

Accordingly, the mechanism of one aspect of the present invention comprises agonism of M4 mAChR.

Mechanism E of Scopolamine

M5 receptors are located on the cell bodies of DA neurons located in the VTA and some on the cholinergic interneurons in the NAc. Studies suggest that systemic administration of a M5 agonist releases DA in NAc by stimulating M5 receptors located on DA neurons in the VTA rather than M5 receptors in the NAc (Threlfell et al. 2010, J. Neuroscience, 30(9):3398-3408).

Without being bound by any theory, the applicant hypothesizes that scopolamine blocks M2/M4 receptors on the cell bodies and terminals of cholinergic neurons projecting from the pedunculopontine tegmental nucleus to the VTA, increasing ACh release in the VTA. ACh released in the VTA stimulates M5 receptors located on DA neurons to release DA in the NAcShell.

Mechanism F of Scopolamine

Nicotinic receptors are located on DA terminals in the NAc. These nicotinic receptors are potent facilitators of DA release.

Without being bound by any theory, the applicant hypothesizes that ACh released by scopolamine stimulates nicotinic receptors to facilitate DA release, thereby further alleviating depression and anxiety.

Accordingly, the mechanism of one aspect of the present invention comprises agonism of nAChR.

Mechanism G of Scopolamine

Nicotinic receptors are also located on GABA-containing interneurons and/or on GABA-containing axonal collaterals of the MSN. Stimulating these nicotinic receptors potently facilitates GABA release, and GABA in turn stimulates GABAergic receptors to inhibit MSN to facilitate behavior and to elevate mood and motivation.

Without being bound by any theory, the applicant hypothesizes that ACh released by scopolamine stimulates nicotinic receptors to release GABA, thereby further alleviating depression and anxiety.

### DEFINITIONS

### Abbreviations

The following list of abbreviations is used throughout this disclosure: NAc—nucleus accumbens; NAcShell—nucleus accumbens shell; NAcCore—nucleus accumbens core; VTA—ventral tegmental area; ACh—acetylcholine; DA—dopamine Glu-Glutamate; GIuR-Glutamate receptor; NMDA—N-Methyl-D-aspartic acid; GABA—γ-Aminobutyric acid; CRF—corticosterone-releasing factor; MSN—GABA-containing medium spiny neurons projecting out of the nucleus accumbens; nAChR—nicotine-sensitive cholinergic receptor; mAChR—muscarinic acetylcholine receptor.

The term “reward” refers to positive feelings, including pleasure, satisfaction, a sense of relief, or sensitivity to a stimulus eliciting pleasure.

The term “hedonic capacity” refers to the range of reward a person or an animal is capable of experiencing.

The term “motivation” generally refers to as an internal drive to engaging in either approach behavior (exploration or reward seeking) or active avoidance behavior (to escape from an aversive situation). Motivation also varies in intensity.

The term “treating” refers to delaying, halting, alleviating, reversing, or preventing the onset of, the progress of, one or more symptoms of the disorder or condition to which the term applies. As used herein, the term “treating” does not imply a cure, permanent or otherwise, for the disorder or condition.

The term “mental and behavioral disorders” includes: (a) deficiencies or disorders related to affect (e.g., mood, feelings of attachment or sympathy or empathy, pleasure or aversion, range of emotion and motivation, sense of personal efficacy); (b) deficiencies or disorders related to cognition (e.g., organization of thoughts, perception of physical senses and situational realities, attention, vigilance, memory, learning, production and understand of language, problem solving, decision making); (c) Maladaptive behaviors or patterns of behavior (e.g., decreased ability to regulate instinctual, habitual, or stereotypic actions, to execute goal-oriented action, to follow through tasks, to care for oneself, to relate to others, to actively cope with or overcome adversity, to act or carry a demeanor appropriate to universal norms or social- cultural- and age-dependent norms); and (d) persistent or excessive distress (e.g., physical or emotional pain, anxiety, discomfort).

The term “mental and behavior disorders” includes: the syndrome or pattern of a primary clinical diagnosis of “mental and behavioral disorders”; conditions co-occurring with a primary clinical diagnosis (i.e., comorbid conditions); and specific conditions occurring in multiple, different clinical diagnoses. Clinical diagnoses of mental and behavioral disorders comprise those indicated in American Psychiatric Association Diagnostic and Statistical Manual of Mental Disorders, Fourth Edition Text Revision (DSM-IV-TR), the World Health Organization's ICD-10 Classification of Mental and Behavioral Disorders, and related diagnoses in their future revisions (e.g., DSM-V, ICD-11). The term “mental and behavior disorders” further include subclinical conditions decreasing or preventing the affected individuals' ability to function optimally in different environments (e.g., in occupation, educational, social, and recreational settings) and/or adversely affecting their general quality of life.

The terms “antagonism” and “agonism” “are used synonymously with the same terms defined by the International Union of Basic and Clinical Pharmacology (IUPHARM) in the publication: “International Union of Pharmacology Committee on Receptor Nomenclaturee and Drug Classification XXXVIII. Update on Terms and Symbols in Quantitative Pharmacology”, Pharmacological Reviews, 2003, 55(4): 597-606 (see below).

The term “selective antagonist” refers to an antagonist with “preferential” binding to the receptor subtype of interest (e.g., M1 mAChR), compared to other subtypes from the same class (e.g., M2-M5 mAChRs), as determined using radioligand binding techniques, with a selective ligand (e.g., [3H]pirenzepine) or a non-selective ligand (e.g., [³H]N-methylscopolamine), in combination with membrane preparations from cells transfected with the gene for the receptor. The potency of an antagonist for the receptor of interest can be expressed in terms of the concentration at which 50% of the radiolabeled ligand is displaced (IC50), or in terms of the dissociation constant K_{d} or Kᵢ. A selective antagonist will have a IC50, K_{d}, or Kᵢ value for the receptor of interest at least 2-fold less than its respective values for other receptor subtypes that are structurally similar to the one of interest (e.g., M1 vs M3). When comparing a binding value of an antagonist to the receptor of interest relative its binding values for other receptor subtypes exhibiting greater structural dissimilarities than the receptor of interest, a 5-fold or 10-fold difference is preferred.

Below are the definitions of terms used to describe drug action from the International Union of Pharmacology adapted in this disclosure (International Union of Pharmacology Committee on Receptor Nomenclaturee and Drug Classification XXXVIII. Update on Terms and Symbols in Quantitative Pharmacology, Pharmacological Reviews, 2003, 55(4): 597-606.)

The term “agonist” refers to a ligand that binds to a receptor and alters the receptor state resulting in a biological response. Conventional agonists increase receptor activity, whereas inverse agonists reduce it.

Receptor activity may be determined by: the proportion of receptor in an active or high-affinity conformation (R*), relative to the receptor in an inactive or low-affinity conformation (R), post-translational modifications (e.g., phosphorylation), or some other mechanism such as subcellular targeting.

Agonists may act by combining either with the same site(s) as the endogenous agonist (primary or orthosteric site) or with a different region of the receptor macromolecule (allosteric or allotopic site).

Agonists in the second category referred to as allosteric (allotopic) activators or allosteric (allotopic) agonists. Some agonists (e.g., glutamate) may only be effective in the presence of another ligand (e.g., glycine in the case of glutamate) that binds to a different site on the receptor macromolecule. Under these circumstances, glutamate is referred to as the primary agonist and glycine as a co-agonist.

The term “agonist [of a receptor]” refers to an administered substance (e.g., a drug) that reduces the action of either an endogenous agonist (e.g., a neurotransmitter or a peptide) of the receptor or an administered drug (an agonist or another antagonist). Several categories of antagonists act at the same receptor macromolecule as the agonist (see below for classification of antagonists).

The term “antagonism” refers to either chemical antagonism or functional antagonism.

The term “chemical antagonism” refers to an antagonist combining with the substance (e.g., a receptor or the neurotransmitter of the receptor) being antagonized.

The term “functional antagonism” refers to antagonism occurring at cellular sites distinct from the receptor mediating the agonist response. Functional antagonism comprises indirect antagonism and physiological antagonism. Indirect antagonism is competition by the inhibitor for the binding site of an intermediate macromolecule that links the binding of the administered agonist to the effect observed (e.g., adrenoceptor antagonist blockade of the actions of tyramine or protein kinase A inhibitors blocking adrenoceptor agonist effects). Physiological antagonism is the action of one agonist exerts an opposite effect to that of the original agonist—usually through a different receptor (e.g., muscarinic agonist inhibition of adrenoceptor-stimulated adenylyl cyclase activity in the heart).

The term “allosteric (allotypic) modulator” refers to a ligand that increases or decreases the action of an (primary or orthosteric) agonist or antagonist by combining with a distinct (allosteric or allotopic) site on the receptor macromolecule.

The terms “allosteric (allotopic) enhancer” or “positive allosteric modulator (PAM)” interchangeably refer to a modulator that enhances orthosteric ligand affinity and/or agonist efficacy while having no effect on its own.

The terms “allosteric (allotopic) antagonist” or “negative allosteric modulator (NAM)” interchangeably refer to a modulator that reduces orthosteric ligand affinity and/or agonist efficacy. Allosteric (allotopic) agonists or activators are ligands that are able to mediate receptor activation in their own right by binding to a recognition domain on the receptor macromolecule that is distinct from the primary (orthosteric) site.

The term “bitopic interaction” refers to binding of a ligand to both the orthosteric and an allosteric site on the same receptor.

The term “neutral allosteric (allotopic) ligand” refers to a ligand that binds to an allosteric site without affecting the binding or function of orthosteric ligands but can still block the action of other allosteric modulators that act via the same allosteric site.

The term “syntopic interaction” refers to an interaction between ligands that bind to the same recognition site, or to recognition sites that overlap, on the receptor macromolecule. This term describes competitive interactions between ligands that bind to the primary (orthosteric) site on a receptor, but need not be restricted to this specific situation. A syntopic interaction can also occur between different ligands that share a similar recognition domain (e.g., a common allosteric site) anywhere on the receptor macromolecule.

The term “allosteric (allotopic) interaction” refers to an interaction between ligands that bind to distinct, non-overlapping, recognition sites on the receptor macromolecule.

The terms “syntopic” and “allotopic” distinguish between interactions that occur at a common (same) site versus interactions that occur between different sites, respectively. The term allotopic is used interchangeably with the term allosteric only when describing cross-interactions between different sites on a receptor macromolecule.

The term “syntopic” refers to interactions at a common site and is not used interchangeably with the term orthosteric; the latter term specifically refers to the primary (endogenous agonist-binding) recognition site on the receptor.

The term “allosteric transition” refers to the isomerization of a receptor macromolecule between multiple conformational states. Different authors have used the term, allosteric, in different ways (see Colquhoun, 1998; Christopoulos and Kenakin, 2002). One common use of the term is to describe any mechanism that involves the isomerization of a receptor between two or more conformational states that can each display a different affinity for a given ligand. A second common use of the term is to explicitly describe an interaction between two topographically distinct recognition sites on a receptor macromolecule in a given conformational state. In order to accommodate both uses, the term allosteric transition is used when describing receptor isomerization mechanisms, and the term allosteric (or allotopic) interaction is used when describing a cross-interaction between multiple ligands concomitantly bound to a receptor macromolecule.

The term “efficacy” refers to the degree to which different agonists produce varying responses, even when occupying the same proportion of receptors. Efficacy is both agonist- and tissue-dependent. The term “intrinsic efficacy” is used when discussing the agonist, rather than the tissue-dependent component of efficacy. The term “efficacy” when used alone refers to the comparative activity of agonists on intact tissues.

The term “full agonist” refers to an agonist that induces maximal response capability of the system (tissue).

If the maximum tissue response is reached at less than full receptor occupancy it results in a so-called spare receptor situation (see below). Several agonists may thus elicit the same maximal response, albeit at different receptor occupancies. They are all full agonists in that experimental system but have different efficacies. This designation of full versus partial agonist is system-dependent, and a full agonist for one tissue or measurement may be a partial agonist in another.

The term “inverse agonist” refers to a ligand that by binding to receptors reduces the fraction of them in an active conformation. This can occur if some of the receptors are in the active form (R*), in the absence of a conventional agonist. If the ligand, combines preferentially with inactive receptors, it will reduce the fraction in the active state. An inverse agonist may combine either with the same site as a conventional agonist, or with a different site on the receptor macromolecule.

The term “partial agonist” refers to an agonist that in a given tissue, under specified conditions, cannot elicit as large an effect (even when applied at high concentration, so that all the receptors should be occupied) as can another agonist, such as a full agonist, acting through the same receptors in the same tissue.

The designation “partial agonist” is system-dependent and a partial agonist in one experimental system may be a full agonist in another (e.g., one in which there were more receptors expressed). Recent advances make it clear that the inability of a particular agonist to produce a maximal response can have several explanations. Perhaps the most important is that not enough of the receptors occupied by the agonist convert to an active form, and the term partial agonist is now sometimes applied to this situation alone. The term “partial agonist” used herein does not apply to this latter scenario.

The distinction between such usages can be illustrated by the action of decamethonium at the neuromuscular junction. Decamethonium cannot match the conductance increase caused by acetylcholine. However, this is not because decamethonium is less able to cause the receptors to isomerize to an active form: rather, the smaller maximal response is largely a consequence of the greater tendency of decamethonium to block the ion channel that is intrinsic to the nicotinic receptor. Hence, decamethonium would not be regarded as a partial agonist with respect to receptor conformational equilibria but would be in the broader sense of the term.

The term “spare receptors” refers to receptors in a pharmacological system in which a full agonist can cause a maximum response when occupying only a fraction of the total receptor population. Thus not all of the receptors in the tissue are required to achieve a maximal response with some high efficacy agonists. This has been amply demonstrated experimentally by Furchgott (1966) and others in that irreversible chemical inactivation of some receptors results in a decrease in agonist potency without a decreased maximal response. At sufficiently high degrees of receptor inactivation, the maximum response even to full agonists is finally reduced. Although all receptors may not be needed for a maximal response, all receptors contribute to the measured responses, thus the potency of full agonists (and often the physiological agonists) is enhanced by the presence of the spare receptors.

In analyzing pharmacological properties of ligands or interpreting results with receptor mutants in heterologous expression systems, which often have very high levels of receptor expression, it is essential to understand and account for the spare receptor phenomenon. Many compounds that are partial agonists in normal tissues are full agonists in expression systems due to the high receptor number (see for example, Brink et al., 2000).

The term “competitive antagonism” refers to antagonism in which the binding of agonist and antagonist is mutually exclusive. This may be because the agonist and antagonist compete for the same binding site or combine with adjacent sites that overlap (syntopic interaction). A third possibility is that different sites are involved but that they influence the receptor macromolecule in such a way that agonist and antagonist molecules cannot be bound at the same time.

The term “reversible competitive antagonism” refers to antagonism in which the agonist and antagonist form only short-lasting combinations with the receptor, so that equilibrium between agonist, antagonist, and receptors is reached during the presence of the agonist, the antagonism will be surmountable over a wide range of concentrations.

The term “irreversible competitive antagonism” refers to antagonism in which an antagonist, when in close enough proximity to their binding site, may form a stable covalent bond with it, and the antagonism becomes insurmountable when no spare receptors remain. More generally, the extent to which the action of a competitive antagonist can be overcome by increasing the concentration of agonist is determined by the relative concentrations of the two agents, by the association and dissociation rate constants for their binding, and by the duration of the exposure to each.

The term “noncompetitive antagonism” refers to antagonism in which the agonist and antagonist is bound to the receptor simultaneously; antagonist binding reduces or prevents the action of the agonist with or without any effect on the binding of the agonist. The usage is limited to the action of blockers on the same receptor as the agonist (such as channel block of the nicotinic receptor).

The term “insurmountable antagonism” refers to antagonism in which the maximum effect of the agonist is reduced by either pretreatment or simultaneous treatment with the antagonist. This can encompass several distinct molecular mechanisms such as: (a) irreversible competitive antagonism; (b) noncompetitive antagonism; and (c) functional antagonism. Determining whether a system is under insurmountable antagonism requires distinguishing between the locus of the action (competitive, noncompetitive, or indirect) and the kinetics of the action (reversible and irreversible) of the ligands involved.

The term “surmountable antagonism” refers to antagonism generally observed with reversible competitive antagonism though it may also occur with chemical antagonism, with irreversible antagonists in the case of spare receptors, or with certain forms of allosteric antagonism.

The placement of therapeutically active agents below into various classes reciting their activity (i.e., full agonists, partial agonists, antagonists, inverse agonists, allosteric modulators, and other classes) and reciting the receptor or other target thereof are not to be taken as exclusive. The placement of these compounds into such classes and the receptor or other target effected can vary depending on the therapeutically active agent involved, the receptors or targets, involved, the concentration of the therapeutically active agent administered, or the presence or absence of other agonists, antagonists, or modulators of the receptors or targets in the particular environment being studied. A therapeutically active compound, for example, can be a full agonist of one receptor and a partial agonist of another receptor. Similarly, a therapeutically active compound can be a full agonist of one receptor and a partial agonist of another receptor. Therefore, such multiple classification does not suggest or imply that the activities of the therapeutically active agent being classified are non-specific or unclear.

In addition to the compounds described below, analogs or derivatives of these compounds having substantially equivalent pharmacological activity with respect to the receptors or targets identified can be used. As used herein, the term “substantially equivalent pharmacological activity” means activity that is at least 80% as much as the parent compound on a molar basis with respect to the specific receptor or target. Typically, the activity is at least 90% as much as the parent compound on a molar basis. Preferably, the activity is at least 95% as much as the parent compound on a molar basis. More preferably, the activity is at least 97.5% as much as the parent compound on a molar basis. Most preferably, the activity is at least 99% as much as the parent compound on a molar basis. The potential changes that can be included in analogs or derivatives include, but are not limited to: replacement of one halogen (chlorine, fluorine, bromine, or iodine) with another halogen; replacing one or more hydrogens with lower alkyl, typically C₁-C₆ alkyl; replacing one lower alkyl residue with another lower alkyl residue; or replacing one or more hydrogens in an amino group with lower alkyl. The term “lower alkyl”, unless further limited, refers to both straight-chain and branched alkyl groups. These derivatives or analogs can be further substituted with one or more groups that do not substantially affect the pharmacological activity of the derivative or analog. Such groups are known in the art.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

The compounds used in methods of the present invention can also include solvates of those compounds. As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

Other compounds that are bioisosteres of the compounds described below can also be used. A bioisostere is a compound that replaces a group present in the original compound with another group that retains the desired biological activity. For example, a pyrrole ring can replace an amide to generate a bioisostere. An ester or a 5-substituted tetrazole can replace a carboxyl group to generate a bioisostere. The methyl group of an ethanoate ester can be replaced with NH2 to generate a bioisostere.

Here Applicant presents a novel method of treatment of depression (e.g., major depression, bipolar disorder), anxiety, and PTSD involving administering a cholinergic muscarinic M1 receptor antagonist (M1 antagonist) alone or together with a cholinomimetic that either potentiates the activities of cholinergic receptors other than the M1 receptor or potentiates the activity of an inhibitor of the acetylcholine degradation enzyme acetylcholinesterase. Such drug combination may also be useful for treating mental and behavioral disorders other than depression and anxiety, including, but not only, other affective disorders, addictive disorders, attention deficit disorder, generalized anxiety disorder, panic disorder, social phobia, eating disorders, obsessive compulsive disorder, post-traumatic stress syndrome, movement disorder, sexual dysfunction, and the like.

The idea behind the invention is to block the cholinergic M1 receptor pathway that subserves depression and anxiety while increasing the activities of cholinergic receptors other than the M1 to facilitate the release of dopamine and GABA. This strategy is modeled after the Applicant's hypothesized mechanism of action of scopolamine, as indicated above.

One particular M1 antagonist is telenzepine (4,9-dihydro-3-methyl-4-[(4-methyl-1-piperazinyl)acetyl]-10H-thieno[3,4-b][1,5]benzodiazepin-10-one). Telenzepine can be administered in doses ranging from 0.1 mg/day to about 20 mg/day. A dose falling within 1 to 10 mg/day is preferred. The present method also can use chemical analogs or enantiomers of telenzepine.

Another M1 antagonist is pirenzepine (5,11-dihydro-11-[4-methyl-1-piperazinyl)acetyl]-6H-pyrido[2,3-b][1,4]benzodiazepine-6-one). Pirenzepine can be administered in doses ranging from 10 mg/day to 200 mg/day, preferably between 20-100 mg/day.

Other M1 antagonists that can be administered include, but not limited to: amytriptyline (3-(10,11-dihydro-5H-dibenzo[a,d]cycloheptene-5-ylidene)-N,N-dimethylpropan-1-amine) administered in doses ranging from 20 to 250 mg/day, preferably 25-150 mg/day; biperiden ((1RS,2SR,4RS)-1-(bicyclo[2.2.1]hept-5-en-2-yl)-1-phenyl-3-(piperidin-1-yl)propan-1-ol) in doses ranging from 2 to 16 mg/day; trihexyphenidyl ((RS)-1-cyclohexyl-1-phenyl-3-(1-piperidyl)propan-1-ol) in doses ranging from 1 to 10 mg/day, preferably 6-10 mg/day; darifenacin ((S)-2-[1-[2-(2,3-dihydrobenzofuran-5-yl)ethyl]pyrrolidin-3-yl]-2,2-diphenyl-acetamide) in doses ranging from 5 to 15 mg/day; dicyclomine (2-(diethylamino)ethyl 1-cyclohexylcyclohexane-1-carboxylate) in doses ranging from 80 to 160 mg/day; and tiotropium (7-[(hydroxidi-2-thienylacetyl)oxy]-9,9-dimethyl-3-oxa-9-azoniatricyclo[3.3.1.02,4]nonane bromide) in intranasal doses of about 18 mcg/day.

M1 antagonists can be used together with a cholinesterase inhibitor. One particular cholinesterase inhibitor is galantamine ((4aS,6R,8aS)-5,6,9,10,11,12-hexahydro-3-methoxy-1′-methyl-4-aH-[1]benzofuro[3a,3,2-ef][2]benzazepin-6-ol). Galantamine is a competitive and reversible cholinesterase inhibitor. It reduces the action of acetylcholinesterase and therefore tends to increase the concentration of acetylcholine in the brain. Galantamine can be taken in doses ranging from 4 to 8 mg/day.

Other important acetylcholinesterase inhibitors that can be used with an M1 antagonist include: tacrine (1,2,3,4-tetrahydroacridin-9-amine) with doses ranging from 10 to 160 mg/day; rivastigmine ((S)-3-[1-(dimethylamino)ethyl]phenyl N-ethyl-N-methylcarbamate) dose ranging from 3 to 12 mg/day; and donepezil ((RS)-2-[(1-benzyl-4-piperidyl)methyl]-5,6-dimethoxy-2,3-dihydroinden-1-one) dose ranging from 5 to 25 mg/day.

Other acetylcholinesterase inhibitors that can be used with an M1 antagonist include huperzine; carbamates including physostigmine, neostigmine, pyridostigmine, ambenonium, demarcarium, and rivastigmine; caffeine, piperidines including donepezil; xanthostigmine; aminobenzoic acid; flavonoid; pyrrolo-isoxazole; edrophonium; ladostigil; ungeremine; lactucopicrin; and coumarin.

Another drug that can be administered together with an M1 antagonist is a non-selective cholinergic muscarinic receptor agonist. One such muscarinic receptor agonist is piracetam (2-oxo-1-pyrrolidineacetamide), which can be administered in doses between 4.8 to 25 mg.

Another muscarinic receptor agonist that can be used with an M1 antagonist is bethanechol (2-(carbamoyloxy)-N,N,N-trimethylpropan-1-aminium). Bethanechol can be used in doses ranging from 10 to 200 mg/day.

Another muscarinic receptor agonist that can be used with an M1 antagonist is cevimeline (2-Methyspiro(1,3-oxathiolane-5,3)quinuclidine). Cevimeline can be used in doses ranging from 30 to 90 mg/day.

M1 antagonists can be used together with a cholinergic nicotinic receptor agonist. One such nicotinic receptor agonist is varenicline (7,8,9,10-Tetrahydro-6,10-methano-6H-pyrazino[2,3-h][3]benzazepine. Varenicline can be used in doses ranging from 0.5 to 2 mg/day.

Other nicotinic receptor agonists that can be used with an M1 antagonist include: galantamine ((4aS,6R,8aS)-5,6,9,10,11,12-hexahydro-3-methoxy-11-methyl-4aH-[1]benzofuro[3a,3,2-ef][2]benzazepin-6-ol) with doses ranging from 4 to 8 mg/day; and nicotine (3-[(2S)-1-methylpyrrolidin-2-yl]pyridine) with doses ranging from 2 to 14 mg/day.

M1 antagonists can be used together with a cholinomimetic named sildenafil (1-[4-ethoxy-3-(6,7-dihydro-1-methyl-7-oxo-3-propyl-1H-pyrazolo[4,3-d]pyrimidin-5-yl)phenylsulfonyl]-4-methylpiperazine). Sildenafil can be used in doses ranging from 25 to 100 mg/day.

Analogs or derivatives of the drugs listed above can also be used.

A skilled practitioner would be expected to determine the precise level of dosing. The ideal dosing would be routinely determined by an evaluation of the patient and the needs of the patient.

The drug combinations should be administered together at the same time. If it is necessary to administer the drugs at different times, the M1 antagonist should be administered before the other drugs (e.g., 1, 2, 3, 4, 5, 6, 8, 10, or 12 hours apart).

The drug combinations may comprise a mixture of the individual drugs. The drug combinations may also comprise a kit consisting of two or more individual drugs.

The drug combinations may be administered through any one of the following routes: oral (“po”), topical contact, intravenous (“iv”), intramuscular (“im”), intraperitoneal (“ip”), intranasal, intralesional, subcutaneous (“sc”), or the implantation of a slow release device such as a mini-osmotic pump. Drugs can be administered parenterally (e.g. intracranial, intraventricular, intraperitoneal, subcutaneous, intradermal, intra-arteriole, intramuscular, intravenous). Other means of administration include, but not limited to, the use of liposomal formulations, transdermal patches, intravenous infusion, etc. In the case of intracranial administration, the targeted site should preferably be aimed at the nucleus accumbens (especially the shell), subgenual prefrontal cortex, or hippocampus.

By far the most convenient route of administration is oral (ingestion). Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

The drugs or drug combinations are intended for use by humans or they can be administered to a mammal other than humans.

Accordingly, one embodiment of this aspect of the present invention is a method for lessening the symptoms of depression and anxiety comprising the step of administering a therapeutically effective quantity of a cholinergic M1 receptor antagonist and a therapeutically effective quantity of one or more cholinomimetic agents to lessen the symptoms of depression and anxiety. Typically, the cholinergic M1 receptor antagonist is selected from the group consisting of telenzepine, amytriptyline, biperiden, trihexyphenidyl, darifenacin, dicyclomine, and tiotropium. This embodiment also includes a composition comprising a therapeutically effective quantity of a cholinergic M1 receptor antagonist, a therapeutically effective quantity of one or more cholinomimetic agents, and, optionally, a pharmaceutically acceptable carrier for use in lessening the symptoms of depression and anxiety. When present, the pharmaceutically acceptable carrier can be selected from the group consisting of a solvent, a buffer, a preservative, a solid filler, an excipient, a diluent, a dispersion medium, a coating, an antibacterial and/or antifungal agent, an isotonic agent, and an absorption-delaying agent. Other pharmaceutically acceptable carriers known in the art can also be used. More than one pharmaceutically acceptable carrier in various combinations known in the art can be employed.

In one alternative, the cholinomimetic comprises an acetylcholinesterase inhibitor. The acetylcholinesterase inhibitor is typically selected from the group consisting of:

(1) a phenanthrene derivative;

(2) tacrine;

(3) a carbamate derivative;

(4) a piperidine derivative;

(5) caffeine;

(6) huperzine;

(7) xanthostigmine;

(8) aminobenzoic acid;

(9) flavonoid;

(10) pyrrolo-oxazole;

(11) edrophonium;

(12) ladostigil;

(13) ungeremine;

(14) lactucopicrin; and

(15) coumarin.

When the acetylcholinesterase inhibitor is a phenanthrene derivative, typically the phenanthrene derivative is galantamine. When the acetylcholinesterase inhibitor is a carbamate derivative, typically the carbamate derivative is selected from the group consisting of rivastigmine, physostigmine, neostigmine, pyridostigmine, ambenonium, and demarcarium. When the acetylcholinesterase inhibitor is a piperidine, typically the piperidine is donezepil.

In another alternative, the cholinomimetic is a cholinergic muscarinic receptor agonist. Typically, the cholinergic muscarinic receptor is selected from the group consisting of piracetam, bethanechol, and cevimeline.

In still another alternative, the cholinomimetic is a cholinergic nicotinic receptor agonist. Typically, the cholinergic nicotinic receptor is selected from the group consisting of varenicline, galantamine, and nicotine.

In still another alternative, the cholinomimetic is sildenafil.

Another aspect of the present invention is directed to methods and compositions employing other therapeutic agents and combinations of therapeutic agents for emulating the theoretical pharmacological effects of the non-selective mAChR antagonist scopolamine.

Accordingly, one embodiment of this aspect of the present invention is a method for treatment of a mental or behavioral disorder comprising administration of a therapeutically effective quantity of an antagonist of muscarinic acetylcholine receptors of subtype M1 (M1 mAChR). Alternatively, the method can comprise administration of a therapeutically effective quantity of an antagonist of muscarinic acetylcholine receptors of subtype M2 (M2 mAChR), or administration of a therapeutically effective quantity of an antagonist of muscarinic acetylcholine receptors of subtype M4 (M4 mAChR).

Yet another embodiment of this aspect of the present invention is a method for treatment of a mental or behavioral disorder comprising administration of a therapeutically effective quantity of an agonist of M1 mAChR. Alternatively, the method can comprise administration of a therapeutically effective quantity of an agonist of muscarinic acetylcholine receptors of subtype M2 (M2 mAChR), administration of a therapeutically effective quantity of an agonist of muscarinic acetylcholine receptors of subtype M3 (M3 mAChR), administration of a therapeutically effective quantity of an agonist of muscarinic acetylcholine receptors of subtype M4 (M4 mAChR), or administration of a therapeutically effective quantity of an agonist of muscarinic acetylcholine receptors of subtype M5 (M5 mAChR).

Yet another embodiment of this aspect of the present invention is a method for treatment of a mental or behavioral disorder comprising administration of a therapeutically effective quantity of both an antagonist and an agonist of M1 mAChr.

Yet another embodiment of this aspect of the present invention is a method for treatment of a mental or behavioral disorder comprising administration of a therapeutically effective quantity of a non-selective antagonist of mAChRs.

Yet another embodiment of this aspect of the present invention is a method for treatment of a mental or behavioral disorder comprising administration of a therapeutically effective quantity of a non-selective agonist of mAChRs.

Yet another embodiment of this aspect of the present invention is a method for treatment of a mental or behavioral disorder comprising administration of a therapeutically effective quantity of an agent to increase acetylcholine (ACh) level.

Yet another embodiment of this aspect of the present invention is a method for treatment of a mental or behavioral disorder comprising administration of a therapeutically effective quantity of an antagonist of glutamate receptors.

Yet another embodiment of this aspect of the present invention is a method for treatment of a mental or behavioral disorder comprising administration of a therapeutically effective quantity of an agonist of nicotinic receptors.

Yet another embodiment of this aspect of the present invention is a method for treatment of a mental or behavioral disorder comprising administration of a therapeutically effective quantity of an agonist of GABA receptors.

Another embodiment of this aspect of the invention comprises a method for treatment of a mental or behavioral disorder comprising administration of a therapeutically effective quantity of an agonist of M2 mAChR to normalize ACh release. Alternatively, the method can comprise administration of a therapeutically effective quantity of an agonist of M4 mAChR to normalize ACh release. In another alternative, the method can comprise administration of a therapeutically effective quantity of an antagonist of glutamate receptors to normalize ACh release. In yet another alternative, the method can comprise administration of a therapeutically effective quantity of an agonist of opiate receptors to normalize ACh release. In still another alternative, the method can comprise administration of a therapeutically effective quantity of an antagonist of CRF receptors to normalize ACh release. In still another alternative, the method can comprise administration of a therapeutically effective quantity of an agent that antagonizes nitric oxide release. In still another alternative, the method can comprise administration of a therapeutically effective quantity of an antagonist of nitric oxide receptors of subtype NR2B. In still another alternative, the method can comprise administration of a therapeutically effective quantity of an agonist of substance P receptors of subtype neurokinin 1 (NK1). In still another alternative, the method can comprise administration of a therapeutically effective quantity of an antagonist of a cytokine or of a receptor of a cytokine. In still another alternative, the method can comprise administration of a therapeutically effective quantity of a modulator of interferon receptors. In still another alternative, the method can comprise administration of a therapeutically effective quantity of an agent that increases the level of P11 protein. In still another alternative, the method can comprise administration of a therapeutically effective quantity of an agonist of a serotonin 5HT1A receptor. In still another alternative, the method can comprise administration of a therapeutically effective quantity of an agonist of a serotonin 5HT1B receptor. In still another alternative, the method can comprise administration of a therapeutically effective quantity of an agent that inhibits serotonin reuptake.

In still another alternative embodiment of this aspect of the invention, the method can comprise administration of a therapeutically effective quantity of an agent that is a selective M1 antagonist. The selective M1 antagonist can be, but is not limited to, MT-7, rMT7,4-DAMP, tripitramine, darifenacin, VU0255035, guanylpirenzepine, AFDX384, pirenzepine, himbacine, telezepine, MT3, AF-DX 116, biperiden, trihexyphenidyl, dicyclomine, tiotropium, or N-methylscopolamine.

In another alternative embodiment of this aspect of the invention, the method can comprise administration of a therapeutically effective quantity of an agent that is a non-selective M1 antagonist. The non-selective M1 antagonist can be, but is not limited to, scopolamine, atropine, pF-HHSiD, dicycloverine, isopropamide, glycopyrrolate, clidinium bromide, doxepin, clozapine, olanzapine, chlorpromazine, thioridazine, pilocarpine, benztropine and benztropine analogs, diphenylpyraline (DPP), ziprasidone, an imidazole derivative that is a non-selective M1 antagonist, or imidafenacin.

In still another alternative embodiment of this aspect of the invention, the method can comprise administration of a therapeutically effective quantity of an agent that is an inverse agonist of the M1 receptor. The inverse agonist of the M1 receptor can be, but is not limited to, AF-DX 116, atropine, N-methylscopolamine, QNB, R-(−) QNB, 4-DAMP, pirenzepine, or trihexyphenidyl.

In still another alternative embodiment of this aspect of the invention, the method can comprise administration of a therapeutically effective quantity of an agent that is a selective partial agonist of the M1 receptor. The partial agonist of the M1 receptor can be, but is not limited to, CCD-0102A or LY593093.

In still another alternative embodiment of this aspect of the invention, the method can comprise administration of a therapeutically effective quantity of a non-selective partial agonist of the M1 receptor. The non-selective partial agonist of the M1 receptor can be, but is not limited to, xanomeline, sabcomeline, oxotremorine, pilocarpine, McN-A-343, milameline, (−)YM796, (±)YM796, or (−)aceclidine.

In still another alternative embodiment of this aspect of the invention, the method can comprise administration of both: (i) a therapeutically effective quantity of a selective partial agonist of the M1 receptor; and (ii) a therapeutically effective quantity of a non-selective partial agonist of the M1 receptor. Suitable selective partial agonists of the M1 receptor and non-selective partial agonists of the M1 receptor are as described above.

In still another alternative embodiment of this aspect of the invention, the method can comprise administration of a selective negative allosteric modulator of the M1 receptor. The selective negative allosteric modulator of the M1 receptor can be, but is not limited to, MT-7; CID-25010775, or tiotropium.

In still another alternative embodiment of this aspect of the invention, the method can comprise administration of a non-selective negative allosteric modulator of the M1 receptor. The non-selective negative allosteric modulator of the M1 receptor can be, but is not limited to, Gö 7874, WIN 51,708, WIN 62,577, eburnamonine, alcuronium, strychnine, vincamine, brucine, N-benzylbrucine, N-chloromethylbrucine, thiochrome, brucine N-oxide, alcuronium, or AC-42.

In still another alternative embodiment of this aspect of the invention, the method can comprise administration of a neutral allosteric modulator of the M1 receptor. The neutral allosteric modulator of the M1 receptor can be, but is not limited to, xanomeline, sabcomeline, oxotremorine, pilocarpine, McN-A-343, milameline, (−) YM796, (±)YM796, or (−)aceclidine.

In still another alternative embodiment of this aspect of the invention, the method can comprise administration of a therapeutically effective quantity of a selective positive allosteric modulator of the M1 receptor. The selective positive allosteric modulator of the M1 receptor can be, but is not limited to, N-desmethylclozapine, AF267B, MT-7, CID-25010775, AC-42, 77-LH-28-1, AC-260584, KT-5823, staurosporine, KT-5823, K-252a, TBPB, LuAE51090, BQCA, KT5720, VU0090157, VU0029767 or ML169 (VU0405652). The method can further comprise administration of a therapeutically effective quantity of one or more of brucine, brucine N-oxide, or clozapine.

In still another alternative embodiment of this aspect of the invention, the method can comprise administration of both: (i) a therapeutically effective quantity of a non-selective orthosteric M1 antagonist; and (ii) a therapeutically effective quantity of a non-selective negative allosteric modulator of the M1 receptor. The non-selective orthosteric M1 antagonist can be, but is not limited to, scopolamine, sabcomeline, atropine, pF-HHSiD, dicycloverine, isoproamide, glycopyrrolate, clidinium, bromide, doxepin, clozapine, olanzapine, chlorpromazine, thioridazine, propantheline, ipratropium, darifenacin, pirenzepine, methoctramine, HHSiD, himbacine, AF-DX 116, and xanomeline. The non-selective negative allosteric modulator of the M1 receptor can be, but is not limited to, allamine, strychnine brucine, KT5720, WIN62,577, WIN51,708, or staurosporine.

In yet another alternative embodiment of this aspect of the invention, the method can comprise administration of: (i) a therapeutically effective quantity of an orthosteric M2 antagonist; (ii) a therapeutically effective quantity of an inverse agonist of the M2 receptor; and (iii) a therapeutically effective quantity of an allosteric modulator of the M2 receptor. The orthosteric M2 antagonist can be, but is not limited to, tripitramine, 4-DAMP, himbacine, AF-DX 116, pirenzepine, piperidinyl, piperidine, propantheline, dexetimide, ipratropium, scopolamine, SCH 57790, atropine, methoctramine, hexocyclium, silahexocyclium, imipramine, hexahydrodifenidol, HHSiD, dicyclomine, p-F-HHSiD, gallamine, lithocholylcholine, evetimide, eburnamonine, thiochrome, vincamine, and alcuronium. The inverse agonist of the M2 receptor can be, but is not limited to, darifenacin, tolterodine, oxybutynin, eburnamonine, thiochrome, vincamine, and alcuronium. The allosteric modulator of the M2 receptor can be, but is not limited to, WIN 51,708, WIN 62,577, Gö 7874, N-benzylbrucine, N-chloromethylbrucine, brucine, gallamine, brucine N-oxide, eburnamonine, thiochrome, vincamine, or alcuronium.

In still another alternative embodiment of this aspect of the invention, the method can comprise administration of a therapeutically effective quantity of an orthosteric M4 agonist. The orthosteric M4 agonist can be, but is not limited to, MT3, darifenacin, himbacine, AF-DX 116, pirenzepine, propantheline, scopolamine, ipratropium, atropine, silahexocyclium, hexocyclium, p-F-HHSiD, hexahydrodifenidol, HHSiD, MT1, methoctramine, MT2, or lithocholylcholine.

In still another alternative embodiment of this aspect of the invention, the method can comprise administration of both: (i) a therapeutically effective quantity of a negative allosteric modulator of the M4 receptor; and (ii) a therapeutically effective quantity of a neutral allosteric modulator of the M4 receptor. The negative allosteric modulator of the M4 receptor can be, but is not limited to, WIN 51,708, WIN 62,577, alcuronium, brucine, and N-benzyl brucine. The neutral allosteric modulator of the M4 receptor can be, but is not limited to, KT 5720, Gö 7874, staurosporine, N-chloromethyl-brucine, brucine N-oxide, and N-benzyl brucine.

In still another alternative embodiment of this aspect of the invention, the method can comprise administration of a therapeutically effective quantity of at least one orthosteric M2 full agonist. The orthosteric M2 full agonist can be, but is not limited to, NNC 11-1585, NNC 11-1607, pentylthio-TZTP, NNC 11-1314, xanomeline, oxotremorine, arecaidine propargyl ester, arecoline, carbachol, methylfurmethide, oxotremorine-M, furmethide, bethanechol, (+)aceclidine, pilocarpine, (−)aceclidine, KT 5823, staurosporine, strychnine, N-benzylbrucine, N-chloromethylbrucine, brucine, brucine N-oxide, dimethyl-W84, W-84, or WDuo3.

In still another alternative embodiment of this aspect of the invention, the method can comprise administration of a therapeutically effective quantity of at least one orthosteric M2 partial agonist. The orthosteric M2 partial agonist can be, but is not limited to, NNC 11-1585, NNC 11-1607, pentylthio-TZTP, NNC 11-1314, xanomeline, oxotremorine, arecaidine propargyl ester, arecoline, carbachol, methylfurmethide, oxotremorine-M, furmethide, bethanechol, (+)aceclidine, pilocarpine, (−)aceclidine, KT 5823, staurosporine, strychnine, N-benzylbrucine, N-chloromethylbrucine, brucine, brucine N-oxide, dimethyl-W84, W-84, or WDuo3.

In still another alternative embodiment of this aspect of the invention, the method can comprise administration of a therapeutically effective quantity of at least one positive allosteric modulator of the M2 receptor. The positive allosteric modulator of the M2 receptor can be, but is not limited to, NNC 11-1585, NNC 11-1607, pentylthio-TZTP, NNC 11-1314, xanomeline, oxotremorine, arecaidine propargyl ester, arecoline, carbachol, methylfurmethide, oxotremorine-M, furmethide, bethanechol, (+)aceclidine, pilocarpine, (−)aceclidine, KT 5823, staurosporine, strychnine, N-benzylbrucine, N-chloromethylbrucine, brucine, brucine N-oxide, dimethyl-W84, W-84, or WDuo3.

In still another alternative embodiment of this aspect of the invention, the method can comprise administration of: (i) a therapeutically effective quantity of an orthostatic M4 full agonist; (ii) a therapeutically effective quantity of an orthosteric partial agonist of the M4 receptor; and (iii) a therapeutically effective quantity of a positive allosteric modulator of the M4 receptor. The orthostatic M4 full agonist can be, but is not limited to, pentylthio-TZTP, NNC 11-1585, NNC 11-1607, NNC 11-1314, arecaidine propargyl ester, arecoline, oxotremorine, oxotremorine-M, methylfurmethide, carbachol, furmethide, bethanechol, or (+)aceclidine. The orthosteric partial agonist of the M4 receptor can be, but is not limited to, xanomeline, sabcomeline, McN-A-343, milameline, pilocarpine, or (−)aceclidine. The positive allosteric modulator of the M4 receptor can be, but is not limited to, strychnine, eburnamonine, vincamine, thiochrome, brucine N-oxide, brucine, N-chloromethyl-brucine, N-benzyl brucine, staurosporine, KT 5823, WDuo3, W-84, or dimethyl-W84.

In yet another alternative embodiment of this aspect of the invention, the method can comprise administration of: (i) a therapeutically effective quantity of an orthostatic M3 full agonist; (ii) a therapeutically effective quantity of an orthosteric partial agonist of the M3 receptor; and (iii) a therapeutically effective quantity of a positive allosteric modulator of the M3 receptor. The orthostatic M3 full agonist can be, but is not limited to, NNC 11-1585, NNC 11-1607, pentylthio-TZTP, arecaidine propargyl ester, arecoline, oxotremorine, oxotremorine-M, (+)aceclidine, bethanechol, carbachol, furmethide, or methylfurmethide. The orthosteric partial agonist of the M3 receptor can be, but is not limited to, xanomeline, sabcomeline, McN-A-343, milameline, pilocarpine, or (−)aceclidine. The positive allosteric modulator of the M3 receptor can be, but is not limited to, WIN 62,577, N-benzylbrucine, brucine N-oxide, or N-chloromethylbrucine.

In yet another alternative embodiment of this aspect of the invention, the method can comprise administration of: (i) a therapeutically effective quantity of an orthostatic M5 full agonist; (ii) a therapeutically effective quantity of an orthosteric partial agonist of the M5 receptor; and (iii) a therapeutically effective quantity of a positive allosteric modulator of the M5 receptor. The orthostatic M5 full agonist can be, but is not limited to, NNC 11-1585, NNC 11-1607, NNC 11-1314, carbachol, or (+)aceclidine. The orthosteric partial agonist of the M5 receptor can be, but is not limited to, sabcomeline, xanomeline, milameline, pilocarpine, McN-A-343, or (−)aceclidine. The positive allosteric modulator of the M5 receptor can be, but is not limited to, brucine N-oxide.

In yet another alternative embodiment of this aspect of the invention, the method can comprise administration of a therapeutically effective quantity of a benztropine compound or its analogs.

In yet another alternative embodiment of this aspect of the invention, the method can comprise administration of a therapeutically effective quantity of a diphenylpiperidine compound. The diphenylpiperidine compound can be, but is not limited to, diphenylpyraline (DPP).

In yet another alternative embodiment of this aspect of the invention, the method can comprise administration of a therapeutically effective quantity of a mixed selective M1/M3 antagonist. The mixed selective M1/M3 agonist can be, but is not limited to, imidafenacin, an imidazole derivative, KRP-197, or bencycloquidium bromide (BCQB).

In yet another alternative embodiment of this aspect of the invention, the method can comprise administration of a therapeutically effective quantity of an antagonist of the metabotropic glutamate receptors (mGluRs) subtype mGluR1.

In yet another alternative embodiment of this aspect of the invention, the method can comprise administration of a therapeutically effective quantity of an antagonist of the metabotropic glutamate receptors (mGluRs) subtype mGluR5.

In still another alternative embodiment of this aspect of the invention, the method can comprise administration of a therapeutically effective quantity of an antagonist of NMDA glutamate receptors.

In still another alternative embodiment of this aspect of the invention, the method can comprise administration of a therapeutically effective quantity of a selective antagonist of opiate receptor subtypes μ or δ.

In still another alternative embodiment of this aspect of the invention, the method can comprise administration of a therapeutically effective quantity of an antagonist of an opiate receptor that is non-subtype-selective.

In still another alternative embodiment of this aspect of the invention, the method can comprise administration of an agonist of galanin receptor subtype 2 (GalR2).

Methods as described above in these embodiments of this aspect of the invention can further comprise the administration of a therapeutically effective quantity of a partial agonist of dopamine D2 receptors. In another alternative, methods as described above can further comprise the administration of a therapeutically effective quantity of an agent inhibiting dopamine reuptake. In still another alternative, methods as described above can further comprise the administration of a therapeutically effective quantity of an agent inhibiting norepinephrine reuptake. In still another alternative, methods as described above can further comprise the administration of a therapeutically effective quantity of an antagonist of norepinephrine α_{2C} receptors. In still another alternative, methods as described above can further comprise the administration of a therapeutically effective quantity of an antagonist of norepinephrine α_{2A} receptors. In still another alternative, methods as described above can further comprise the administration of a therapeutically effective quantity of an antagonist of norepinephrine α₂ receptors.

Methods as described above in these embodiments of this aspect of the invention can be employed to augment the effect of another medication as described below.

The methods as described above in these embodiments of this aspect of the invention can further comprise the administration of a therapeutically effective quantity of a compound from one of the following classes of psychotropic medications: antidepressants, antipsychotics, compounds demonstrating antipsychotic properties, mood stabilizers, stimulants, anxiolytics, hypnotics/sedatives, entheogens or nootropics (e.g., cognitive enhancer), anti-ADHD agents, antiaddictives, euphoriants, antidementia agents, depressants, anticonvulsants, analgesics, anesthetics (general, local), antimigraine agents, anorectics, antiparkinson's agents, neuroprotectives, orexigenics, or wakefulness-promoting agents.

When the additional psychotropic agent is an antidepressant, the antidepressant can be an antidepressant from the class of selective serotonin reuptake inhibitors (SSRI), serotonin-norepinephrine reuptake inhibitors (SNRI), serotonin-norepinephrine-dopamine reuptake inhibitors (SNDRI), norepinephrine reuptake inhibitors (NRI), dopamine reuptake inhibitors (DRI), norepinephrine-dopamine reuptake inhibitors (NDRI), noradrenergic and specific serotonergic antidepressant (NaSSA), monoamine oxidase inhibitors (MAOI), and tricyclic compounds. When the antidepressant is a SSRI, the SSRI can be, but is not limited to, dapoxetine, femoxetine, indalpine, zimelidine, fluoxetine, citalopram, paroxetine, sertraline, alaproclate, fluvoxamine, etoperidone, or escitalopram. When the antidepressant is a SNRI, the SNRI can be, but is not limited to, desvenlafaxine, duloxetine, milnacipran, venlafaxine, levomilnacipran, sibutramine, bicifadine, SEP-227162, or edivoxetine (LY 2216684). When the antidepressant is a SNDRI, the SNDRI can be, but is not limited to, sibutramine, amitifadine, tesofensine, bicifadine, RG7166, SEP-227162, SEP-225289, or Tedatioxetine. When the antidepressant is a NRI, the NRI can be, but is not limited to, atomoxetine/tomoxetine (Strattera), mazindol (Mazanor, Sanorex), reboxetine (Edronax, Vestra), or viloxazine (Vivalan). When the antidepressant is a DRI, the DRI can be, but is not limited to, amineptine (Survector, Maneon, Directim), armodafinil (Nuvigil), benzatropine/benztropine (Cogentin), bupropion (Wellbutrin, Zyban), dexmethylphenidate (Focalin), esketamine (Ketanest S), etybenzatropine/ethybenztropine (Panolid, Ponalid, Ponalide), fencamfamine (Glucoenergan, Reactivan), fencamine (Altimina, Sicoclor), ketamine (Ketalar, Ketaset, Ketanest, Ketaject), lefetamine (Santenol), medifoxamine (Cledial), mesocarb (Sidnocarb, Sydnocarb), methylphenidate (Ritalin, Concerta), modafinil (Provigil), nefopam (Acupan), nomifensine (Merital), pipradrol (Meretran), prolintane (Promotil, Katovit), pyrovalerone (Centroton, Thymergix), tiletamine (Telazol, Rompun), or tripelennamine (Pyribenzamine). When the antidepressant is a NRDI, the NRDI can be, but is not limited to, buproprion. When the antidepressant is a NaSSA, the NaSSA can be, but is not limited to, aptazapine (CGS-7525A), esmirtazapine (ORG-50,081), mianserin (Bolvidon, Norval, Tolvon), mirtazapine (Remeron, Avanza, Zispin), or setiptiline (Tecipul). When the antidepressant is a MAOI, the MAOI can be, but is not limited to, isocarboxazid, nialamide, phenelzine, tranylcypromine, iproniazide, iproclozide, moclobemide, or toloxatone. When the antidepressant is a tricyclic compound, the tricyclic compound can be, but is not limited to, amineptine, amitriptyline, clomipramine, desipramine, doxepin, dothiepin, imipramine, nortriptyline, protriptyline, trimipramine, or amoxapine.

When the additional psychotropic agent is an antipsychotic, the antipsychotic can be a phenothiazine with an aliphatic side-chain, a phenothiazine with a piperazine structure, a phenothiazine with a piperidine structure, a butyrophenone derivative, an indole derivative, a thioxanthene derivative; a diphenylbutylpiperidine derivative, a diazepine, an oxazepine, a thiazepine, an oxepine, a benzamide, lithium, a typical antipsychotic, an atypical antipsychotic, a dopamine system stabilizers or an antipsychotic that is classified as an antipsychotic of another type. When the antipsychotic is a phenothiazine with an aliphatic side-chain, the phenothiazine with an aliphatic side-chain can be, but is not limited to, chlorpromazine, levomepromazine, promazine, acepromazine, triflupromazine, cyamemazine, or chlorproethazine. When the antipsychotic is a phenothiazine with a piperazine structure, the a phenothiazine with a piperazine structure can be, but is not limited to, dixyrazine, fluphenazine, perphenazine, prochlorperazine, thiopropazate, trifluoperazine, acetophenazine, thioproperazine, butaperazine, or perazine. When the antipsychotic is a phenothiazine with a piperidine structure, the phenothiazine with a piperidine structure can be, but is not limited to, periciazine, thioridazine, mesoridazine, or pipotiazine. When the antipsychotic is a butyrophenone derivative, the butyrophenone derivative can be, but is not limited to, haloperidol, trifluperidol, melperone, moperone, pipamperone, bromperidol, benperidol, droperidol, fluanisone, or azaperone. When the antipsychotic is an indole derivative, the indole derivative can be, but is not limited to, oxypertine, molindone, sertindole, or ziprasidone. When the antipsychotic is a thioxanthene derivative, the thioxanthene derivative can be, but is not limited to, flupentixol, clopenthixol, chlorprothixene, thiothixene, or zuclopenthixol. When the antipsychotic is a diphenylbutylpiperidine derivative, the diphenylbutylpiperidine derivative can be, but is not limited to, fluspirilene, pimozide, or penfluridol. When the antipsychotic is a diazepine, oxazepine, thiazepine, or oxepine, the antipsychotic can be, but is not limited to, loxapine, clozapine, olanzapine, quetiapine, asenapine, or clotiapine. When the antipsychotic is a benzamide compound, the benzamide compound can be, but is not limited to, sulpiride, sultopride, tiapride, remoxipride, amisulpride, veralipride, or levosulpiride. When the antipsychotic is a typical antipsychotic, the typical antipsychotic can be, but is not limited to, haloperidol; prothipendyl; a benzamide compound such as: levosulpiride, nemonapride, sulpiride, sultopride, tiapride, or veralipride; a butyrophenone compound such as: azaperone, benperidol, bromperidol, droperidol, fluanisone, haloperidol, lenperone, moperone, pipamperone, spiperone, timiperone, or trifluperidol; a diphenylbutylpiperidine compound such as: clopimozide, fluspirilene, penfluridol, or pimozide; a phenothiazine compound such as: acepromazine, acetophenazine, butaperazine, carphenazine, chloracizine, chlorproethazine, chlorpromazine, cyamemazine, dixyrazine, fluacizine, fluphenazine, levomepromazine/methotrimeprazine, mesoridazine, perazine, periciazine, perphenazine, piperacetazine, pipotiazine, prochlorperazine, promazine, promethazine, propiomazine, sulforidazine, thiethylperazine, thiopropazate, thioproperazine, thioridazine, trifluoperazine, or triflupromazine; a thioxanthene compound such as: chlorprothixene, clopenthixol, flupentixol, thiothixene, or zuclopenthixol; or a tricyclic compound such as: amoxapine, butaclamol, fluotracen, loxapine, metitepine/methiothepin, octoclothiepin, or trimipramine. When the antipsychotic is an atypical antipsychotic, the atypical antipsychotic can be, but is not limited to, amisulpride; remoxipride; a butyrophenone compound such as: cinuperone or setoperone; a benzo(iso)oxazolepiperidine compound such as: iloperidone, ocaperidone, paliperidone, or risperidone; a benzo(iso)thiazolepiperazine compound such as: lurasidone, perospirone, revospirone, tiospirone, or ziprasidone; a diphenylbutylpiperazine compound such as amperozide; a phenylpiperazine compound such as: aripiprazole, bifeprunox, elopiprazole, or umespirone; a tricyclic compound such as: asenapine, carpipramine, clocapramine, clotiapine, clozapine, fluperlapine, gevotroline, metitepine/methiothepin, mosapramine, ndmc, olanzapine, piquindone, quetiapine, tenilapine, or zotepine; or other atypical antipsychotics such as: blonanserin, cariprazine, molindone, pimavanserin, roxindole, sarizotan, sertindole, or spiramide. When the antipsychotic is an antipsychotic that is classified as an antipsychotic of another type, the antipsychotic that is classified as an antipsychotic of another type can be, but is not limited to, prothipendyl, risperidone, mosapramine, zotepine, aripiprazole, paliperidone, iloperidone, or amperozide.

When the additional psychotropic agent is a compound demonstrating antipsychotic properties, the compound demonstrating antipsychotic properties can be, but is not limited to, cannabidiol, D-cycloserine, lithium, mifepristone, oxypertine, reserpine, rimcazole, secretin, talnetant, tetrabenazine, vabicaserinazacyclonol, tetrabenazine, compounds exhibiting metabotropic glutamate receptor 2 agonism, compounds exhibiting glycine transporter 1 inhibition, and L-theanine.

When the additional psychotropic agent is a mood stabilizer, the mood stabilizer can be, but is not limited to, an anticonvulsant, lithium, an atypical antipsychotic with mood stabilizing effects, or another agent with mood stabilizing effects. The anticonvulsant can be, but is not limited to, valproic acid, divalroex sodium, sodium valproate, lamotrigine, carbamazepine, oxcabazepine, riluzole, gabapentin, or topiramate. The atypical antipsychotic with mood stabilizing effects can be, but is not limited to, risperidone, olanzapine, quetiapine, paliperidone, or ziprasidone. The additional agent with mood stabilizing effects can be, but is not limited to, omega-3 fatty acids or L-methylfolate.

When the additional psychotropic agent is a stimulant, the stimulant can be, but is not limited to: theobromine; theophylline; nicotinic receptor agonists; amphetamine; norepinephrine reuptake inhibitors (NRI); norepinephrine-dopamine reuptake inhibitors (NDRI); methylphenidate; modafinil (and modafinil prodrugs and derivatives, including adrafinil and armodafinil); ampakines; yohimbine; amphetamine, dexamphetamine; dextromethamphetamine; methylphenidate; pemoline; fencamfamin; fenozolone; atomoxetine; fenetylline; dexmethylphenidate; lisdexamfetamine; xanthine derivatives; caffeine; propentofylline; meclofenoxate; pyritinol; piracetam; deanol; fipexide; citicoline; oxiracetam; pirisudanol; linopirdine; nizofenone, aniracetam, acetylcarnitine; idebenone; prolintane; pipradrol; pramiracetam; adrafinil; or vinpocetine.

When the additional psychotropic agent is an anxiolytic, the anxiolytic can be, but is not limited to, an anxiolytic from the class of benzodiazepine and its derivatives; an anxiolytic from the class of diphenylmethane and its derivatives; an anxiolytic from the class of carbamate and its derivatives; an anxiolytic from the class of dibenzo-bicyclo-octadinene and its derivatives, an anxiolytic from the class of azaspirodecanedione and its derivatives; or other anxiolytics, including SSRIs, azapirones, pregabalin, and other drugs including BNC210, CL-218,872, L-838,417, and SL-651,498. When the anxiolytic is an anxiolytic from the class of benzodiazepine and its derivatives, the anxiolytic can be, but is not limited to, diazepam, chlordiazepoxide, medazepam, oxazepam, potassium clorazepate, lorazepam, adinazolam, bromazepam, clobazam, ketazolam, prazepam, alprazolam, halazepam, pinazepam, camazepam, nordazepam, fludiazepam, ethyl loflazepate, etizolam, clotiazepam, cloxazolam, tofisopam, lorazepam combinations, hydroxyzine, captodiame, or hydroxyzine derivatives. When the anxiolytic is an anxiolytic from the class of carbamate and its derivatives, the anxiolytic can be, but is not limited to, meprobamate, emylcamate, mebutamate, or meprobamate combinations. When the anxiolytic is an anxiolytic from the class of dibenzo-bicyclo-octadinene and its derivatives, the anxiolytic can be, but is not limited to, benzoctamine. When the anxiolytic is an anxiolytic from the class of azaspirodecanedione and its derivatives, the anxiolytic can be, but is not limited to, buspirone. When the anxiolytic is another anxiolytic, the anxiolytic can be, but is not limited to, mephenoxalone, gedocarnil, or etifoxine.

When the additional psychotropic agent is a hypnotic or sedative, the hypnotic or sedative can be, but is not limited to, a hypnotic or sedative from the class of barbiturates, a hypnotic or sedative from the class of aldehydes and derivatives thereof, a hypnotic or sedative from the class of benzodiazepine derivatives, a hypnotic or sedative from the class of piperidinedione derivatives, a hypnotic or sedative from the class of benzodiazepine related drugs, a hypnotic or sedative from the class of melatonin receptor agonist, or other hypnotics or sedatives. When the hypnotic or sedative is a barbiturate, the barbiturate can be, but is not limited to, pentobarbital, amobarbital, butobarbital, barbital, aprobarbital, secobarbital, talbutal, vinylbital, vinbarbital, cyclobarbital, heptabarbital, reposal, methohexital, hexobarbital, thiopental, ethallobarbital, allobarbital or proxibarbal. When the hypnotic or sedative is an aldehyde or derivative thereof, the hypnotic or sedative can be, but is not limited to, chloral hydrate, chloralodol, acetylglycinamide chloral hydrate, dichloralphenazone, or paraldehyde. When the hypnotic or sedative is a benzodiazepine derivative, the benzodiazepine derivative can be, but is not limited to, diazepam (valium), from flurazepam, nitrazepam, flunitrazepam, estazolam, triazolam, lormetazepam, temazepam, midazolam, brotizolam, quazepam, loprazolam, doxefazepam, cinolazepam, clonazepam, alprazolam, or climazolam. When the hypnotic or sedative is a piperidone derivative, the piperidone derivative can be, but is not limited to, glutethimide, methyprylon, or pyrithyldione. When the hypnotic or sedative is a benzodiazepine related drug, the benzodiazepine related drug can be, but is not limited to, zopiclone, zolpidem, zaleplon, or eszopiclone. When the hypnotic or sedative is a melatonin receptor agonist, the melatonin receptor agonist can be, but is not limited to, melatonin or ramelteon. When the hypnotic or sedative is another hypnotic or sedative, the hypnotic or sedative can be, but is not limited to, methaqualone, clomethiazole, bromisoval, carbromal, scopolamine, propiomazine, triclofos, ethchlorvynol, valerianae radix, hexapropymate, bromides, apronal, valnoctamide, methylpentynol, niaprazine, dexmedetomidine, detomidine, medetomidine, xylazine, romifidine, or metomidate. In another alternative, the hypnotic or sedative can be a hypnotic and sedative combination that can be, but is not limited to, meprobamate combinations, methaqualone combinations, methylpentynol combinations, clomethiazole combinations, emepronium combinations, or dipiperonylaminoethanol combinations.

Methods as described above can be used to treat a number of mental or behavioral disorders, including, but not limited to disorders with the symptoms of:

(1) mood disorder;

(2) depressive disorder;

(3) dysthymic disorder (dysthymia or dysthymic episode);

(4) major depressive disorder (or episode);

(5) minor, mild, or moderate depressive disorder (or episode);

(6) disruptive mood dyregulation disorder;

(7) premenstrual dysphoric disorder;

(8) mixed anxiety/depression;

(9) substance-induced depressive disorders;

(10) depressive disorders associated with medical conditions;

(11) bipolar disorders;

(12) cyclothymic disorders;

(13) bipolar disorders due to medical conditions;

(14) substance-induced bipolar disorders;

(15) disorders usually first diagnosed in infancy, childhood, or adolescence (or neurodevelopmental disorders);

(16) mental retardation;

(17) learning disorders (including mathematics disorder, reading disorder, disorder of written expression, and learning disorder not otherwise specified);

(18) motor skills disorders or developmental coordination disorders;

(19) communication disorders (including expressive language disorder, phonological disorder, mixed receptive-expressive language disorder, stuttering, and communication disorder not otherwise specified);

(20) pervasive developmental disorders (including Asperger's disorder, autistic disorder, childhood disintegrative disorder, Rett's disorder, pervasive developmental disorder, and atypical autism not otherwise specified);

(21) attention-deficit and disruptive behavior disorders (including attention-deficit (with or without hyperactivity) disorder, conduct disorder, oppositional defiant disorder, and disruptive behavior disorder not otherwise specified);

(22) feeding and eating disorders of infancy or early childhood (including feeding disorder of infancy or early childhood, pica, and rumination disorder);

(23) tic disorders (including chronic motor or vocal tic disorder, tourette's disorder, and tic disorder not otherwise specified);

(24) other disorders of infancy, childhood, or adolescence (including selective mutism, separation anxiety disorder, reactive attachment disorder of infancy or early childhood, stereotypic movement disorder, and disorder of infancy, childhood, or adolescence not otherwise specified);

(25) mental and behavioral disorders due to medical conditions, including catatonic disorder;

(26) substance-related disorders;

(27) substance use disorder (including substance abuse, dependence, and addiction);

(28) alcohol use disorder (including alcohol abuse, dependence, and addiction);

(29) alcohol-induced disorders (including anxiety disorder; mood disorder; amnestic disorder; dementia; psychotic disorder; sexual dysfunction; sleep disorder; intoxication; intoxication delirium; withdrawal; and delirium; and related disorder not otherwise specified);

(30) amphetamine (or amphetamine-like substances) use disorder (including amphetamine or amphetamine-like substance abuse, dependence, and addiction);

(31) amphetamine (or amphetamine-like substances)-induced disorders (anxiety disorder; mood disorder; psychotic disorder; sexual dysfunction; sleep disorder; intoxication; intoxication delirium; withdrawal; and related disorders not otherwise specified);

(32) caffeine use disorders (including caffeine abuse, dependence, and addiction);

(33) caffeine-induced disorders (including anxiety disorder; sleep disorder; intoxication; withdrawal; difficulties in concentration; and related disorders not otherwise specified);

(34) cannabis use disorders (including cannabis abuse, dependence, and addiction);

(35) cannabis-induced disorders (including anxiety disorder; psychotic disorder; intoxication; intoxication delirium; chronic psychosis; withdrawal; and related disorders not otherwise specified);

(36) cocaine use disorders (including cocaine abuse, dependence, and addiction);

(37) cocaine-induced disorders (including anxiety disorder; mood disorder; psychotic disorder; sexual dysfunction; sleep disorder; intoxication; intoxication delirium; withdrawal; and related disorders not otherwise specified);

(38) hallucinogen use disorders (including hallucinogen abuse, dependence, and addiction);

(39) hallucinogen-induced disorders (including anxiety disorder; mood disorder; psychotic disorder; intoxication; intoxication delirium; perception disorder; withdrawal; and related disorders not otherwise specified);

(40) inhalant use disorders (including inhalant abuse, dependence, and addiction);

(41) inhalant-induced disorders (including anxiety disorder; mood disorder; dementia; psychotic disorder; intoxication; intoxication delirium; withdrawal; and related disorders not otherwise specified);

(42) opioid use disorders (including opioid abuse, dependence, and addiction);

(43) opioid-induced disorders (including mood disorder; psychotic disorder; sexual dysfunction; sleep disorder; intoxication; intoxication delirium; withdrawal; and related disorders not otherwise specified);

(44) nicotine (or tobacco) use disorders (including nicotine abuse, dependence, and addiction);

(45) nicotine-induced disorders (including nicotine withdrawal syndrome: dysphoric or depressed mood; insomnia; irritability, frustration, or anger; anxiety; difficulty concentrating; restlessness; decreased heart rate; increased appetite or weight gain; and withdrawal);

(46) phencyclidine (or phencyclidine-like substances) use disorders (including abuse, dependence, and addiction toward such substances);

(47) phencyclidine (or phencyclidine-like substances)-induced disorders (including anxiety disorder; mood disorder; psychotic disorder; intoxication; intoxication delirium; withdrawal; and other related disorder not otherwise specified);

(48) sedative, hypnotic, or anxiolytic use disorders (including abuse, dependence, and addiction toward such substances);

(49) sedative, hypnotic, or anxiolytic-induced disorders (including anxiety disorder; mood disorder; persisting amnestic disorder; persisting dementia; psychotic disorder; sexual dysfunction; sleep disorder; intoxication; intoxication delirium; withdrawal; withdrawal delirium; and related disorder not otherwise specified).

(50) polysubstance related disorder (including abuse, dependence, and addiction toward multiple substances);

(51) schizophrenia, schizophrenia spectrum disorder, and other psychotic disorders;

(52) schizophrenia;

(53) psychotic disorders other than schizophrenia (including symptoms of schizophreniform disorder, schizoaffective disorder, and delusional disorder, schizotypal disorder, persistent delusional disorders, acute and transient psychotic disorder, induced delusional disorder, schizoaffective disorder, other nonorganic psychotic disorders, and unspecified non-organic psychosis);

(54) anxiety disorders;

(55) generalized anxiety disorder;

(56) panic disorder (with or without agoraphobia);

(57) agoraphobia (without history of panic disorder);

(58) specific phobias;

(59) social phobias;

(60) obsessive compulsive disorder;

(61) posttraumatic stress disorder;

(62) acute stress disorder;

(63) anxiety disorders related to general medical conditions;

(64) substance-induced anxiety disorders;

(65) somatoform disorders;

(66) somatization disorder;

(67) undifferentiated somatoform disorder;

(68) conversion disorder;

(69) pain disorder (including pain associated with psychological factors and/or medical conditions;

(70) hypochondriasis;

(71) factitious disorders;

(72) sexual and gender identity disorders (including sexual desire disorders; sexual arousal disorders; orgasmic disorders; sexual pain disorders; sexual dysfunction due to general medical conditions; substance-induced sexual dysfunction; paraphilias; and gender identity disorder);

(73) eating disorders;

(74) anorexia nervosa;

(75) bulimia nervosa;

(76) obesity;

(77) substance-induced obesity (including antipsychotic medication-induced obesity);

(78) sleep disorders;

(79) primary sleep disorder (including hypersomnia; insomnia; and dyssomnia not otherwise specified);

(80) parasomnias (including nightmare disorder; sleep terror disorder; and parasomnia not otherwise specified);

(81) sleep disorders due to general medical conditions (including hypersomnia type; insomnia type; mixed type; and parasomnia type);

(82) sleep disorder induced by substances (including insomnia, hypersomnia, parasomnia, or mixed type);

(83) impulse control disorders (including intermittent explosive disorder; kleptomania; pathological gambling; pyromania; trichotillomania; and impulse-control disorder due to other causes);

(84) adjustment disorders (including anxiety; depressed mood; disturbance of conduct; mixed anxiety and depressed mood; mixed disturbance of emotions and conduct; other symptoms of depression);

(85) personality disorders;

(86) paranoid personality disorder, schizoid personality disorder, and schizotypal personality disorder;

(87) antisocial personality disorder, borderline personality disorder, histrionic personality disorder, and narcissistic personality disorder;

(88) avoidant personality disorder, dependent personality disorder, and obsessive-compulsive personality disorder;

(89) excessive shyness (with reference to age, cultural, and/or social norms);

(90) excessive self-consciousness (with reference to age, cultural, and/or social norms); and

(91) excessive behavioral inhibition (or lack of spontaneity) (with reference to age, cultural, and/or social norms).

Another aspect of the present invention is a pharmaceutical composition. In general, the pharmaceutical composition comprises:

(1) a therapeutically effective quantity of one or more therapeutic agents affecting brain reward circuitry as described above; and

(2) a pharmaceutically acceptable carrier.

When the pharmacologically active compound in a pharmaceutical composition according to the present invention possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, di nitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The amount of a given pharmacologically active agent that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Optimal dosages for a given set of conditions can be ascertained by those skilled in the art using conventional dosage-determination tests in view of the experimental data for an agent.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions, or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, buffers, preservatives, solid fillers, excipients, diluents, dispersion media, coatings, antibacterial and/or antifungal agents, isotonic agents, absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Pharmaceutical compositions according to the present invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Pharmaceutical compositions according to the present invention are usually administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated, as described above. Typically, such clinical parameters are behavioral in nature and can be determined by tests known in the art.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a pharmacologically active agent according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

In some alternatives, a prodrug of one or more therapeutically active agents as described above can be employed. The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference. In general, prodrugs can be classified into two major types, based on their cellular sites of bioactivation into the final active drug form, with Type I being those that are bioactivated intracellularly (e.g., anti-viral nucleoside analogs, lipid-lowering statins), and Type II being those that are bioactivated extracellularly, especially in digestive fluids or the systemic circulation (e.g., etoposide phosphate, valganciclovir, fosamprenavir, antibody- gene- or virus-directed enzyme prodrugs [ADEP/GDEP/VDEP] for chemotherapy or immunotherapy). Both types can be further categorized into subtypes, i.e. Type IA, IB and Type IIA, IIB, and IIC based on whether or not the intracellular bioactivating location is also the site of therapeutic action, or the bioactivation occurs in the gastrointestinal (GI) fluids or systemic circulation. Type IA prodrugs include many antimicrobial and chemotherapy agents (e.g., 5-fluorouracil). Type IB agents rely on metabolic enzymes, especially in hepatic cells, to bioactivate the prodrugs intracellularly to active drugs. Type II prodrugs are bioactivated extracelluarly, either in the milieu of GI fluids (Type IIA), within the systemic circulation and/or other extracellular fluid compartments (Type IIB), or near therapeutic target tissues/cells (Type IIC), relying on common enzymes such as esterases and phosphatases or target directed enzymes. Importantly, prodrugs can belong to multiple subtypes (i.e., mixed-type). A mixed-type prodrug is one that is bioactivated at multiple sites, either in parallel or sequential steps. Many ADEPs, VDEPs, GDEPs and nanoparticle- or nanocarrier-linked drug moieties can be sequential mixed-type prodrugs. Bioactivation of prodrugs can occur by many reactions, including bioactivation by esterases, hydrolysis, bioactivation by decarboxylases, bioactivation by phosphatases, bioactivation by deacetylases, bioactivation by N-dealkylases, and many other reactions.

In certain other embodiments of the invention, one or more therapeutically active agents of the present invention are associated with a carrier substance such as a compound or molecule (e.g., an antibody, antibody fragment, receptor, or other specific carrier), to facilitate the transport of the one or more active compounds to the intended site of action.

Methods for binding such therapeutically active agents to an individual carrier substance are known in the art. Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail. Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

The individual carrier substances can be, but are not limited to, antibodies, hormones, receptor agonists or antagonists, or receptors. As used herein, unless further defined or limited, the term “antibody” encompasses both polyclonal and monoclonal antibodies, as well as genetically engineered antibodies such as chimeric or humanized antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. Receptors are well known in the art and include G-protein coupled receptors (GPCRs). G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCRs are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors. Agonists and antagonists specifically binding these receptors can be used as individual carrier substances; suitable receptors, agonists, or antagonists can be selected based on their specificity and the location of the receptors in particular cells or tissues.

In some alternatives, therapeutic agents employed in compositions or methods according to the present invention can be chosen, modified, or conjugated in order to enhance passage through the blood-brain barrier. The blood-brain barrier arises because capillaries supplying the brain are lined with tight-fitting cells that do not contain pores and the capillaries are coated with a fatty layer formed from nearby cells, providing an extra fatty barrier through which drugs must cross to enter the brain or central nervous system. Therefore, drugs entering the brain or central nervous system have to dissolve through the cell membranes of the capillaries and also through the fatty cells coating the capillaries. This leads to the result that drugs that are polar or that have an excessive number or concentration of polar groups enter the brain or central nervous system poorly. In order to enter the brain or central nervous system, drugs need to have a minimum number of polar groups, have the polar groups present in the form of prodrugs in which at least some of the polar groups are temporarily masked, or be formulated so that they can cross the blood-brain barrier with the aid of carrier proteins.

U.S. Pat. No. 6,573,292 by Nardella, U.S. Pat. No. 6,921,722 by Nardella, U.S. Pat. No. 7,314,886 to Chao et al., and U.S. Pat. No. 7,446,122 by Chao et al., which disclose methods of use of various pharmacologically active agents and pharmaceutical compositions in treating a number of diseases and conditions and methods of determining the therapeutic effectiveness of such pharmacologically active agents and pharmaceutical compositions, are all incorporated herein by this reference.

The invention is illustrated by the following Example. The Example is included for illustrative purposes only and is not intended to limit the invention.

### Example

FIG. 1 is a diagram illustrating the interactions of the brain reward circuitry.

FIG. 2 is a graph illustrating the effect of antagonism of M1 mAChR and agonism of mAChR in the NAcShell. The discrete trial current-threshold intracranial self-stimulation paradigm were used to assess the effect of cholinergic drugs infused directly into the NAcShell on reward, independent of performance. See below and Markou and Koob (1991) for detailed description of the paradigm. Thresholds measured in a rat vary little in the 4 pre-drug days (<7% of the mean or baseline). The non-selective mAChR agonist arecoline dose-dependently elevates threshold (0.01 M, +11%; 1.0 M, +57.5%, N=2). Pirenzepine dihydrochloride (Sigma-Aldrich, Saint Louis, Mo.), a selective M1 mAChR antagonist, dose-dependently lowers threshold (0.1 mM, −7%; 100 mM, 14%, N=2). The drugs were infused into the NAcShell at a steady rate by reversed microdialysis over the hour required to complete threshold testing. The study clearly indicates that antagonism of M1 mAChR has a rewarding and mood elevating effect, and suggest that mAChR stimulation, including the M1 receptors, may do the opposite, by producing anhedonia.

FIG. 3 is a graph illustrating swimming time following injections of cholinergic drugs in NAc (black bars), compared to Ringer (white bars). (A) Local injection of arecoline, a muscarinic agonist, decreased swim time; the animals quickly gave up (40 μg, −41%, n=8, t=3.53, p<0.01; and 80 μg, −66%, n=6, t=4.12, p<0.01). (B) Pirenzepine, an M1 antagonist, increased swimming escape attempts in the manner of an antidepressant, but note this is a local injection (17.5 μg, n=8, +39%, t=3.63, p<0.01; 35 μg, +40%, n=9, t=7.15, p<0.001). (C) Local scopolamine, a mixed M1 and M2 antagonist, increased swim time (0.5 μg, n=10, N.S.; 1.0 μg, +50%, n=10, t=12.74, p<0.001). (D) Local gallamine, an M2 antagonist, decreased swim time (0.13 μg/side, −21%, n=7, t=3.72, p<0.01; 0.44 μg/side, −37%, n=8, t=2.95, p<0.05; 0.88 μg/side, −36%, n=7, t=4.64, p<0.01). Swimming time following Ringer is normalized to 100%, with each bar representing the mean of the normalized responses across subjects. Swimming time following a drug injection is expressed as a percentage of the swimming time following vehicle injection on a different day. Significant changes in the within-subject response to drug injections are indicated with asterisks (* p<0.05, ** p<0.01, *** p<0.001).

Table 1 shows the effects of infusion into the NAcShell mAChR agonists and antagonists versus Ringer on swimming time. Rats were placed in the 600 sec (10 min) swim test following injections of drugs in the NAcShell. Mean swimming times are shown in the table. When not swimming, rats floated or made minimal forepaw movements to keep their noses above water. Each rat received one drug counterbalanced with Ringer on a separate day. The shorter control swim times for the gallamine group, relative to groups that received other drugs, may have been due to a seasonal difference. Control injections, shown in the last line, were made 2 mm dorsal to the accumbens (Pirenzepine Ctrl). Asterisks indicate significant difference between drug and Ringer (* p<0.05; ** p<0.01; *** p<0.001).

TABLE 1

| | Dose | | Ringer | Drug | Time diff | Percent |
|---|---|---|---|---|---|---|
| Treatments | (μg/side) | N | (sec) | (sec) | (sec) | change |
| Arecholine | 40.0 | 8 | 397 ± 49 | 235 ± 17** | −162 | −41% |
| (non-selective | 80.0 | 6 | 318 ± 65 | 109 ± 34** | −209 | −66% |
| mAChR agonist) | | | | | | |
| Pirenzepine | 17.5 | 8 | 346 ± 45 | 480 ± 38** | +134 | +39% |
| (selective M₁ | 35.0 | 9 | 295 ± 29 | 412 ± 26*** | +117 | +40% |
| antagonist) | | | | | | |
| Scopolamine | 0.5 | 10 | 350 ± 38 | 306 ± 44 | −44 | −12% |
| (non-selective | 1.0 | 10 | 360 ± 24 | 541 ± 13*** | +181 | +50% |
| mAChR | | | | | | |
| antagonist) | | | | | | |
| Gallamine | 0.13 | 7 | 196 ± 20 | 155 ± 26** | −41 | −21% |
| (M₂-preferring | 0.44 | 8 | 236 ± 35 | 148 ± 24* | −88 | −37% |
| antagonist) | 0.88 | 7 | 196 ± 20 | 155 ± 26** | −41 | −36% |
| Pirenzepine Ctrl | 17.5 | 10 | 337 ± 27 | 301 ± 34 | −36 | −10% |

FIG. 4 illustrates the effects of cholinergic drugs on locomotor activity in a photocell cage. Activity in the 10 min following drug injection in the NAc (black bars) is expressed as a percentage of control injections normalized to 100% (white bars). The muscarinic agonist arecoline injected in NAc significantly decreased locomotor activity by reducing rearing and exploratory behavior (40 μg, n=6, * p<0.01). Responses to M1 and M2 antagonists were not significantly different from Ringer (pirenzepine, 35 μg, n=6, N.S.; scopolamine, 1 μg, n=4, N.S.; gallamine, 0.88 μg, n=7, p<0.08).

FIG. 5 illustrates the effect of antagonism of mAChR and agonism of M2 mAChR in NAcShell on ACh efflux. To determine if the M2 drugs act presynaptically on autoreceptors controlling ACh release, freely moving rats received infusions of an M2 agonist or antagonist in the NAc for 20 min by reverse dialysis while extracellular ACh was measured. The M2 agonist, oxotremorine (4 mM perfusate inside the probe) decreased extracellular ACh to 55% of baseline in the hour following the infusion (n=4, F(9,27)=5.57, p<0.001). The M2 antagonist, gallamine at 1 mM probe concentration greatly increased ACh to a level 437% of baseline (n=5, F9,36=5.42, p<0.001). The effect was short lasting. ACh returned to baseline 20 min following the infusion. Scopolamine at 10 mM, to antagonize both M1 and M2 receptors, increased ACh 220% of baseline for more than 100 min (n=5, F9,36=9.23, p<0.001).

FIG. 6 illustrates the effects of local fluoxetine administration on ACh outflow in the NAc during the pretest and the swim tests. * p<0.05; † p<0.001, compared to Ringer. The ACh levels are expressed as percentages of baseline and not in pmole, because absolute levels were not recorded in this experiment. ACh levels during and following 1.0 mM fluoxetine infusion were substantially lower than levels recorded at similar time points during either Ringer infusion or during the pretest (p<0.05, two-way ANOVAs followed by Bonferroni post-hoc tests). Moreover, the swimming time during fluoxetine (329±45 sec) was 18.9% greater than during Ringer (277±44 sec, n=8; one-tail: t=1.89, p<0.005, two-tail: t=2.36, p<0.007). Two-way repeated measures ANOVA indicated significant effects of time (F(19,399)=22.70, p<0.0001) and treatment (F(2,399)=13.66, p<0.0001) on ACh outflow (% of baseline) in NAc, as well as a treatment by time interaction (F(38,399)=6.45, p<0.0001). On test Day 1, ACh decreased below baseline during the second half of the swimming session and remained suppressed during the next 30 min when the rats were removed from the water (p<0.05). On subsequent days (Day 2 or Day 3), during Ringer infusion, ACh again decreased half way into the swim test, but unlike the previous day, ACh remained suppressed for only 15 min after rats were removed from the water (p<0.05). On the days rats received the fluoxetine treatment, ACh dramatically decreased half way into the fluoxetine infusion and remained strongly suppressed below baseline during rest of the observation period (p<0.05). Between-group comparisons using Bonferroni's tests revealed that changes in extracellular ACh during the Ringer day and the pretest day were not different from each other.

Table 2 shows immobility, swimming, and climbing scores of rats during fluoxetine administration (either on Day 2 or Day 3) compared to scores during Ringer administration (either on Day 2 or Day 3) following the initial swim on Day 1. Fluoxetine was counter-balanced by Ringer on alternate days. Fluoxetine (0.2 mM, 0.5 mM, and 0.75 mM) was infused bilaterally in the NAc in three separate groups of rats (n=8-9 per group) either 24 hours or 48 hours following the initial swim on Day 1. Each group of rats received only one dose of fluoxetine, and fluoxetine was counterbalanced with Ringer in the same rat between Day 2 and Day 3. Thus, half of the subjects within each group received one of the three doses of fluoxetine on Day 2 and Ringer on Day 3 of the swim test; the other half in each group received the respective dose of fluoxetine and Ringer in the reserved order (i.e., fluoxetine on Day 3 and Ringer on Day 2). Data from both groups were combined and analyzed together. When examining the data in this manner, only the middle dose (5 mM) show a significant increase in the total escape responses (i.e., swimming plus climbing scores), relative to Ringer (*p<0.05, compared to Ringer).

TABLE 2

| | | | | | | | | | Swimming + Climbing | |
|---|---|---|---|---|---|---|---|---|---|---|
| Fluoxetine (FLU) | | | | | | | | | (total escape | |
| on Day 2 or Day 3 | | | Immobility | | Swimming | | Climbing | | attempts) | |
| Dose | | N | FLU | Ringer | FLU | Ringer | FLU | Ringer | FLU | Ringer |
| 0.2 | mM | 8 | 53 ± 12 | 62 ± 10 | 14 ± 7 | 6 ± 3 | 52 ± 10 | 47 ± 11 | 66 ± 12 | 52 ± 9 |
| 0.5 | mM | 9 | 51 ± 8 | 59 ± 9 | 7 ± 3 | 4 ± 2 | 61 ± 9 | 56 ± 9 | 68 ± 8* | 60 ± 9 |
| 0.75 | mM | 9 | 53 ± 7 | 61 ± 6 | 10 ± 4 | 7 ± 3 | 57 ± 9 | 51 ± 6 | 67 ± 7 | 58 ± 6 |

Table 3 shows immobility, swimming, and climbing scores of rats during fluoxetine administration on Day 3 compared to scores during Ringer on Day 2 following the initial swim on Day 1. Data from animals receiving fluoxetine on Day 3 and Ringer on Day 2 were analyzed separately and the results are presented here. This secondary analysis was done to control for the possibility that fluoxetine could have affected swimming behavior the next day. For this secondary analysis, we hypothesized that the total escape responses would be greater during fluoxetine than during Ringer if fluoxetine was given on Day 3 (but not on Day 2). Consistent with this hypothesis, two-way ANOVA revealed that fluoxetine given on Day 3 decreased immobility scores and increased the total escape response scores, compared to Ringer given on Day 2 (*p<0.05, compared to Ringer).

TABLE 3

| | | | | | | | | | Swimming + Climbing | |
|---|---|---|---|---|---|---|---|---|---|---|
| Fluoxetine (FLU) | | | | | | | | | (total escape | |
| on Day 3 | | | Immobility | | Swimming | | Climbing | | attempts) | |
| Dose | | N | FLU | Ringer | FLU | Ringer | FLU | Ringer | FLU | Ringer |
| 0.2 | mM | 5 | 45 ± 13 | 58 ± 14 | 14 ± 8 | 6 ± 4 | 60 ± 15 | 53 ± 17 | 74 ± 13* | 58 ± 15 |
| 0.5 | mM | 5 | 39 ± 10* | 49 ± 13 | 7 ± 6 | 3 ± 2 | 74 ± 10 | 66 ± 12 | 81 ± 9* | 69 ± 12 |
| 0.75 | mM | 5 | 53 ± 7* | 68 ± 10 | 10 ± 4 | 3 ± 1 | 57 ± 9* | 48 ± 10 | 73 ± 11* | 51 ± 10 |

Table 4 shows immobility, swimming, and climbing scores of rats during fluoxetine administration on Day 2 compared to scores during Ringer on Day 3 following the initial swim on Day 1. Data from animals receiving fluoxetine on Day 2 and Ringer on Day 3 were analyzed separately and the results are presented here. Immobility and escape attempt scores were not different between Day 2 during fluoxetine administration versus Day 2 during Ringer administration. One explanation for the lack of difference in immobility and escape attempt scores between the effects of Day 2 fluoxetine and Day 3 Ringer could be that the fluoxetine treatment given on Day 2 resulted in an improvement in behavioral depression that lasted for 24 h or more, such that when tested again the following day (Day 3), the same animals on Ringer emitted increased escape efforts as they did the day before during fluoxetine. Such lasting effects of local fluoxetine could have occurred due to the residual presence of either fluoxetine or its metabolite and/or to long-lasting changes in the function of the local neural circuitry. Another explanation could be that fluoxetine's action in the accumbens on Day 2 prevented the animals from acquiring a conditioned behaviorally depressed response to the swim test the following day.

TABLE 4

| | | | | | | | | | Swimming + Climbing | |
|---|---|---|---|---|---|---|---|---|---|---|
| Fluoxetine (FLU) | | | | | | | | | (total escape | |
| on Day 2 | | | Immobility | | Swimming | | Climbing | | attempts) | |
| Dose | | N | FLU | Ringer | FLU | Ringer | FLU | Ringer | FLU | Ringer |
| 0.2 | mM | 3 | 66 ± 30 | 68 ± 16 | 15 ± 17 | 6 ± 8 | 39 ± 12 | 36 ± 11 | 54 ± 30 | 43 ± 5 |
| 0.5 | mM | 4 | 68 ± 9 | 72 ± 14 | 6 ± 3 | 4 ± 3 | 45 ± 12 | 44 ± 12 | 51 ± 9 | 48 ± 14 |
| 0.75 | mM | 4 | 62 ± 11 | 53 ± 7 | 14 ± 7 | 13 ± 4 | 44 ± 10 | 54 ± 7 | 58 ± 11 | 67 ± 7 |

FIG. 7 shows the effects of chronic systemic fluoxetine administration on basal extracellular ACh in the NAc. The data suggest that basal ACh was elevated two weeks after the preswim in rats injected daily with saline (‡ p=0.06). The group treated daily with fluoxetine had a normalized basal ACh level. A Chi-square test indicated that chronic fluoxetine and chronic saline had opposing effects on basal ACh (p<0.0001, χ2 for independence). Daily subcutaneous injection of fluoxetine over 14 days counteracted the rise in basal extracellular ACh that otherwise followed a swim two weeks earlier. Basal extracellular ACh level tended to rise following a swim in the control group that received chronic, daily saline treatment (after pretest/before treatment: n=4, 1.52±0.3 pmol; after 14 days of treatment: 4.22±1.47 pmol; t=2.1, p=0.06). However, no change in basal extracellular ACh occurred follow chronic fluoxetine treatment (after pretest/before treatment: n=4, 2.80±0.9 pmol; after 14 days of treatment: 2.37±2.01 pmol, n.s.). A chi-square test further suggested that chronic fluoxetine and chronic saline treatment had opposing effects on the basal extracellular ACh levels, i.e., saline allowed it to rise, whereas fluoxetine kept it stable (p<0.0001, χ2 for independence). The total escape efforts, as indicated by the aggregate scores of swimming plus climbing, in the two treatment groups showed significant differences: the swimming plus climbing scores were higher following chronic fluoxetine treatment than following control chronic saline treatment (saline: 42±11; fluoxetine: 55±7; t=3.3, p<0.05). As expected, the immobility scores following chronic fluoxetine treatment (n=3, 65±7) showed the opposite trend; they were lower than the scores seen following chronic saline treatment (n=3, 77±11; t=3.3, p<0.05).

FIG. 8 is a schematic diagram illustrating the mechanisms of antidepressant action of scopolamine in the nucleus accumbens. In addition to blocking the M1 receptors [mechanism A], scopolamine also blocks M2 and M4 receptors on cholinergic interneurons to disinhibit ACh release [mechanism A]. Elevated ACh increases DA and GABA releases and decreases glutamate release to further alleviate depression and anxiety. ACh released by scopolamine stimulates M2 and M4 receptors on glutamate afferent terminals to inhibit glutamate release, thereby reducing NMDA receptor stimulation and activity in the medium spiny neurons (MSN) to further alleviate depression and anxiety [mechanism B]. ACh released by scopolamine stimulates M3 receptors on dopamine terminals to increase DA release [mechanism C]. ACh released by scopolamine also stimulates MM4 receptors on the MSN projecting to the ventral tegmental area (VTA) to inhibit these MSN, resulting in disinhibition of DA release in the nucleus accumbens [mechanism D]. Scopolamine blocks M2 and M4 receptors on the cell bodies and terminals of cholinergic neurons projecting from the pedunculopontine tegmental nucleus to the VTA, increasing ACh release in the VTA. ACh released in the VTA stimulates M5 receptors located on DA neurons to release DA in the nucleus accumbens [mechanism E]. ACh released by scopolamine stimulates nicotinic receptors (non 7 subtype, especially the 6-containing subtype) on DA terminals to facilitate DA release [mechanism F]. Finally, ACh released by scopolamine stimulates nicotinic receptors on GABA interneurons and on the MSN GABA collaterals to release GABA, and increased GABA release in turn directly inhibits MSN, thereby further alleviating depression and anxiety [mechanism G].

This example confirms the efficacy of scopolamine and other agents that can normalize neurotransmission in the nucleus accumbens, especially in the posterior medial subregion of the nucleus accumbens (the NAc shell) and thus normalize and stabilize the activity of the brain reward circuitry. In particular, these agents have the effect of agonizing and/or antagonizing one or more subtypes of muscarinic acetylcholine receptors (mAChR), agonizing one or more subtypes of nicotinic acetylcholine receptors (nAChR), and normalizing the release of acetylcholine (ACh) in the NAcShell.

### ADVANTAGES OF THE INVENTION

The present invention provides more effective and efficient methods and compositions for treating a number of mental and behavioral diseases and conditions treatable by the modulation of the brain reward circuitry. These methods and compositions are effective in treating these conditions, are well-accepted, and do not cause significant side effects.

Methods according to the present invention possess industrial applicability for the preparation of a medicament for the treatment of a number of diseases and conditions, and possess industrial applicability as pharmaceutical compositions.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug or combination of drugs.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method for lessening the symptoms of depression comprising the step of administering a therapeutically effective quantity of a cholinergic M1 receptor antagonist and a therapeutically effective quantity of one or more cholinomimetic agents to lessen the symptoms of depression.

2. The method of claim 1 wherein the cholinergic M1 receptor antagonist is selected from the group consisting of telenzepine, pirenzepine, amytriptyline, biperiden, trihexyphenidyl, darifenacin, dicyclomine, and tiotropium.

3. The method of claim 1 wherein the cholinomimetic comprises an acetylcholinesterase inhibitor.

4. The method of claim 3 wherein the acetylcholinesterase inhibitor is selected from the group consisting of:
(a) a phenanthrene derivative;
(b) tacrine;
(c) a carbamate derivative;
(d) a piperidine derivative;
(e) caffeine;
(f) huperzine;
(g) xanthostigmine;
(h) aminobenzoic acid;
(i) flavonoid;
(j) pyrrolo-oxazole;
(k) edrophonium;
(l) ladostigil;
(m) ungeremine;
(n) lactucopicrin; and
(o) coumarin.

5. The method of claim 4 wherein the acetylcholinesterase inhibitor is a phenanthrene derivative and the phenanthrene derivative is galantamine.

6. The method of claim 4 wherein the acetylcholinesterase inhibitor is a carbamate derivative and the carbamate derivative is selected from the group consisting of rivastigmine, physostigmine, neostigmine, pyridostigmine, ambenonium, and demarcarium.

7. The method of claim 4 wherein the acetylcholinesterase inhibitor is a piperidine and the piperidine is donezepil.

8. The method of claim 1 wherein the cholinomimetic is a cholinergic muscarinic receptor agonist.

9. The method of claim 8 wherein the cholinergic muscarinic receptor is selected from the group consisting of piracetam, bethanechol, and cevimeline.

10. The method of claim 1 wherein the cholinomimetic is a cholinergic nicotinic receptor agonist.

11. The method of claim 10 wherein the cholinergic nicotinic receptor is selected from the group consisting of varenicline, galantamine, and nicotine.

12. The method of claim 1 wherein the cholinomimetic is sildenafil.

13. A pharmaceutical composition comprising:
(a) a therapeutically effective quantity of a cholinergic M1 receptor antagonist;
(b) a therapeutically effective quantity of one or more cholinomimetic agents; and
(c) optionally, a pharmaceutically acceptable carrier.

14. The pharmaceutical composition of claim 13 wherein the cholinergic M1 receptor antagonist is selected from the group consisting of telenzepine, pirenzepine, amytriptyline, biperiden, trihexyphenidyl, darifenacin, dicyclomine, and tiotropium.

15. The pharmaceutical composition of claim 13 wherein the cholinomimetic comprises an acetylcholinesterase inhibitor.

16. The pharmaceutical composition of claim 15 wherein the acetylcholinesterase inhibitor is selected from the group consisting of:
(a) a phenanthrene derivative;
(b) tacrine;
(c) a carbamate derivative;
(d) a piperidine derivative;
(e) caffeine;
(f) huperzine;
(g) xanthostigmine;
(h) aminobenzoic acid;
(i) flavonoid;
(j) pyrrolo-oxazole;
(k) edrophonium;
(l) ladostigil;
(m) ungeremine;
(n) lactucopicrin; and
(o) coumarin.

17. The pharmaceutical composition of claim 16 wherein the acetylcholinesterase inhibitor is a phenanthrene derivative and the phenanthrene derivative is galantamine.

18. The pharmaceutical composition of claim 16 wherein the acetylcholinesterase inhibitor is a carbamate derivative and the carbamate derivative is selected from the group consisting of rivastigmine, physostigmine, neostigmine, pyridostigmine, ambenonium, and demarcarium.

19. The pharmaceutical composition of claim 16 wherein the acetylcholinesterase inhibitor is a piperidine and the piperidine is donezepil.

20. The pharmaceutical composition of claim 13 wherein the cholinomimetic is a cholinergic muscarinic receptor agonist.

21. The pharmaceutical composition of claim 20 wherein the cholinergic muscarinic receptor is selected from the group consisting of piracetam, bethanechol, and cevimeline.

22. The pharmaceutical composition of claim 13 wherein the cholinomimetic is a cholinergic nicotinic receptor agonist.

23. The pharmaceutical composition of claim 22 wherein the cholinergic nicotinic receptor is selected from the group consisting of varenicline, galantamine, and nicotine.

24. The pharmaceutical composition of claim 13 wherein the cholinomimetic is sildenafil.

25. The pharmaceutical composition of claim 13 wherein the composition comprises the pharmaceutically acceptable carrier.

26. The pharmaceutical composition of claim 25 wherein the pharmaceutically acceptable carrier is selected from the group consisting of a solvent, a buffer, a preservative, a solid filler, an excipient, a diluent, a dispersion medium, a coating, an antibacterial and/or antifungal agent, an isotonic agent, and an absorption-delaying agent.
